(19) United States
(12) Patent Application Publication
(10) Pub. No.: US 2021/0071183 A1
(43) Pub. Date: Mar. 11, 2021

(54) ZINC ASSOCIATED TREATMENT FOR AND DIAGNOSIS OF CACHEXIA

(71) Applicant: THE TRUSTEES OF COLUMBIA UNIVERSITY IN THE CITY OF NEW YORK, New York, NY (US)

(72) Inventors: Swarnali ACHARYYA, New York, NY (US); Anup K. BISWAS, New York, NY (US); Wanchao MA, New York, NY (US); Courtney COKER, New York, NY (US); Gang WANG, New York, NY (US)

(21) Appl. No.: 16/975,803

(22) PCT Filed: Jan. 24, 2019
(86) PCT No.: PCT/US2019/014907

§ 371 Date: Aug. 26, 2020

Related U.S. Application Data
(60) Provisional application No. 62/635,198, filed on Feb. 26, 2018.

(51) Int. Cl. C12N 15/113; A61P 35/04; A61P 21/06; A61K 31/444; A61K 31/47; A61K 31/145; A61K 31/197; A61K 48/00

(52) U.S. Cl. CPC C12N 15/1138; A61P 35/04; A61P 21/06; A61K 48/005; A61K 31/47; A61K 31/145; A61K 31/197; A61K 31/444

(57) ABSTRACT

The present invention provides methods of diagnosing and treating cancer-induced cachexia using a zinc transporter as a biomarker and a therapeutic target. The method for diagnosing cachexia includes monitoring Zip 14-mediated zinc accumulation in the patient's muscle. The method for treating cachexia includes administering a pharmaceutical composition to reduce the Zip 14-mediated zinc accumulation in the patient's muscle.

### PRIORITY

This application claims the benefit of U.S. provisional application 62/635,198 filed Feb. 26, 2018, the entire content of which is expressly incorporated by reference herein.

### FIELD OF THE INVENTION

The present invention provides methods for diagnosing and treating cancer-induced cachexia to improve patient survival. The method of treatment generally relates to a temporary reduction of zinc in the patient. Specifically, this includes administering a pharmaceutical composition to reduce Zip14-mediated zinc accumulation in the patient's muscle. The method for diagnosing cachexia is based on monitoring Zip14-mediated zinc accumulation, including monitoring an expression level of Zip14, a loss of myosin heavy chain, and a reduction of muscle-cell differentiation in the patient's muscle.

### BACKGROUND OF THE INVENTION

Over 90% of cancer-related deaths occur due to metastasis. Lethality from metastasis can be attributed to the invasion and growth of metastatic cells within different organs and the release of secreted soluble proteins, exosomes, and metabolites by metastatic tumors to affect the organs. Tumor-secreted factors can induce a complex metabolic syndrome of extensive muscle damage and weakness, a phenomenon known as cachexia. Metastatic cancer patients experience severe loss of skeletal muscle mass and function which undermines the effectiveness of cancer therapies. Cachexia significantly shortens the survival of cancer patients, since cachectic cancer patients often become too weak to tolerate standard doses of cancer therapies, and those with wasting of diaphragm and cardiac muscles often die due to respiratory and cardiac failure.

A reduction in muscle size also known as muscle atrophy, i.e., a process characterized by marked deterioration of cellular organelles, cytoplasm and proteins in muscles, and is a characteristic of cancer cachexia. Enhanced breakdown of muscle proteins can be accompanied by decreased synthesis, which contributes to the altered muscle homeostasis and muscle mass loss in cancer cachexia. Although cachexia is a key indicator of poor prognosis in cancer patients, the underlying molecular mechanisms of muscle wasting remain poorly understood.

Various biomarkers for diagnosing or monitoring the disease state and progression of cachexia have been disclosed, such as amino acid metabolites or cachexia-associated protein (Assadi-Porter et al, US2009104596; Akerblom et al., U.S. Pat. No. 5,834,192). Klickstein et al. (WO 2017081624) discloses the uses of myostain antagonists for the treatment of cancer cachexia. Wischhusen et al. (WO 2015144855 A1) discloses an antibody against growth and differentiation factor 15 (GDF-15) for treating cancer cachexia. Chen et al. (US 2015015832 A1) discloses a method of suppressing cancer cachexia by administering inhibitors of histone deacetylases (HDAC). However, the targeted pathway to treat the complex metabolic syndrome of cachexia is under characterized. Currently there are no effective treatments and drugs for treating cachexia.

Zinc is an essential trace mineral for normal growth and immune functions as well as the activity of many transcription factors and enzymes, which is regulated by zinc transporters to control zinc influx and efflux between extracellular and intracellular compartments and to modulate the zinc concentration and distribution. Zinc levels in the human body are adjusted properly to maintain the cellular processes and biological responses, since zinc deficiency or excessive zinc absorption can disrupt zinc homeostasis and affects biological functions. (Hara et al., Physiological roles of zinc transporters: molecular and genetic importance in zinc homeostasis, The Journal of Physiological Sciences, March 2017, vol 67, issue 2, page 238-301). Excess zinc accumulation has been observed in cachectic muscles in animal models and patients. It has been hypothesized that systemic zinc dyshomeostasis is relevant to cancer cachexia and zinc redistribution is mediated by acute phase response (Siren et al., Systemic zinc redistribution and dyshomeostasis in cancer cachexia. Journal of Cachexia, Sarcopenia and Muscle 1, 23-33, 2010). However, the mechanism of driving zinc accumulation and the consequence of muscle zinc overload during cancer metastasis have not been studied.

The present invention now uncovers a connection between accumulation of certain metals in muscle and muscle wasting in order to provide a new way to treat cachexia in cancer patients.

### SUMMARY OF THE INVENTION

The present invention provides a method for treating cachexia induced by cancer. The present invention also provides a diagnostic method to predict susceptibility or progression of cachexia to improve survival of cancer patients.

The present invention discloses a method for treating or suppressing cachexia in a patient to increase the survival of the patient. This is achieved by reducing bioavailable zinc in the patient to reduce, inhibit or prevent zinc accumulation in the patient's muscle. This can be achieved by administering a pharmaceutical composition to the patient in an effective amount to reduce a Zip14-mediated zinc accumulation in the patient's muscle. The method may further comprise a step of administering a pharmaceutical composition in an effective amount to reduce a loss of myosin heavy chain in the patient's muscle or a step of administering a pharmaceutical composition in an effective amount to promote muscle-cell differentiation in the patient's muscle.

In one aspect, the pharmaceutical composition comprises a zinc chelating agent and a muscle-specific targeting agent. In addition to administering a zinc chelating agent, the method for treating cachexia may further comprise a step of restricting zinc uptakes in the patient's diet. When the cachexia is caused by cancer, these steps are conducted prior to or immediately after the administration of a cancer treating drug.

In one embodiment, the pharmaceutical composition of treating cachexia comprises an inhibitor of a Zip14 protein, wherein the inhibitor of the Zip14 protein is an antagonist of the Zip14 protein. In another embodiment, the pharmaceutical composition of treating cachexia comprises a nucleic acid which is used to reduce or eliminate the expression of Zip14 in the patient's muscle, wherein the nucleic acid is a short hairpin RNA, a short interfering RNA, or a nucleic acid for gene editing.

The present invention also provides a method for diagnosing a development or a progression of cachexia in a patient comprising monitoring Zip14-mediated zinc accumulation in the patient's muscle, wherein the cachexia is induced by cancer. In one aspect, the method may further comprise a step of monitoring an expression level of Zip14 in the patient's muscle, a step of monitoring a loss of myosin heavy chain in the patient's muscle, or a step of monitoring a reduction of muscle-cell differentiation in the patient's muscle.

Furthermore, the present invention discloses a method for monitoring the development or progression of cachexia in a patient using Zip14 as a biomarker, which comprises detecting an increased-level of Zip14 protein in the patient or by detecting an increased-level of Zip14-mediated zinc accumulation in the patient's muscle. The development or progression of cachexia can be reduced or inhibited by administering an inhibitor of a Zip14 protein to the patient, this improving the patient's quality of life and chance for survival.

The details of the preferred embodiments of the present invention are set forth in the accompanying figures and detailed description herein. Once these details of the invention are known, numerous additional innovations and changes will become obvious and implementable to one of ordinary skill in the art.

### BRIEF DESCRIPTION OF THE FIGURES

Further features of the inventive concept, its nature and various advantages will become more apparent from the following detailed description, taken in conjunction with the accompanying figures:

FIG. 1 (a) through (f) shows analysis results of Zip14 upregulation in cachectic muscles from multiple mouse models of metastasis-induced cachexia and from human cachectic cancer patients.

FIG. 2 (a) through (o) shows schematic representation of tumor induction and metastatic progression in four metastatic models of lung cancer.

FIG. 3 (a) through (j) shows the analysis results of Zip14-mediated zinc uptake in muscles promoting metastatic-cancer-induced cachexia.

FIG. 4 (a) through (y) shows the analysis results of Zip14-WT and Zip14-KO to demonstrate that Zip14 loss reduces metastatic-cancer-induced muscle atrophy.

FIG. 5 (a) through (j) shows the analysis results of non-tumor-bearing control mice (Con) or mice bearing 4T1 or C26m2 metastases to demonstrate that Zip14-mediated zinc accumulation blocks muscle-cell differentiation and induces myosin heavy chain loss.

FIG. 6 (a) through (y) shows the analysis results of myosin heavy chain loss induced by Zip14-mediated zinc uptake in muscle cells.

FIG. 7 (A) through (D) shows the analysis results of the Lewis Lung carcinoma (LLC) mouse model of lung cancer metastasis.

FIG. 8 shows the analysis results of the Pan02 mouse model of pancreatic cancer metastasis indicating the induction of Zip14 with cachexia.

FIGS. 9 (A) and (B) shows the analysis results of zinc chelation treatments for healthy or C26m2 tumor bearing mice treated with regular or zinc-enriched water with zinc chelator injection.

### DETAILED DESCRIPTION OF THE INVENTION

Throughout this description, the preferred embodiments and examples provided herein should be considered as exemplary, rather than as limitations, of the present invention.

The present invention provides methods for diagnosing and treating cancer-induced cachexia to predict susceptibility or progression of cachexia and to improve survival of cancer patients by temporarily reducing zinc in the patient so that zinc cannot be uploaded into the patient's muscle. As zinc is an essential element that is needed by the human body, the reduction or suppression of zinc is maintained temporarily when cancer treatments are administered. There are a number of ways to reduce zinc content but preferably this includes the administration of a zinc chelator. One or more zinc chelators can be administered along with, just prior to or immediately after the administration of a cancer treating drug. Zinc chelating agents or chelators are generally known, e.g., in various references such as US patent publication 20140303081-A1, U.S. Pat. No. 9,320,736, PCT application WO 2013182254 A1, U.S. Pat. No. 6,166,071, Laskaris (Laskaris et al., Administration of Zinc Chelators Improves Survival of Mice Infected with Aspergillus fumigatus both in Monotherapy and in Combination with Caspofungin, Antimicrobial Agents and Chemotherapy, October 2016, volume 60, number 10) and Drobinskaya (Drobinskaya et al., Diethyldithiocarbamate-mediated zinc ion chelation reveals role of Cav2.3 channels in glucagon secretion, Biochimica et Biophysica Acta, 1853 (2015), page 953-964). Laskaris discloses seven zinc chelators, i.e. 1,10-phenanthroline, N,N,N′,N′-tetrakis(2-pyridylmethyl) ethane-1,2-diamine (TPEN), clioquinol (5-chloro-7-iodo-quinolin-8-ol), DEDTC (sodium diethyldithiocarbamate trihydrate), DTPA (diethylene triamine pentaacetic acid), EDDA (ethylenediamine-N,N′-diacetic acid), and EDTA (ethylenediaminetetraacetic acid). Laskaris also discloses that these zinc chelators can be used individually or in combination. Drobinskaya discloses the use of diethyldithiocarbamate (DEDTC) as a chelating agent for zinc to study the function of Cav2.3 channels, such as using three DEDTC injections at 0.025 mg/g body weight each at three time points in 14-17 week old mice.

It is also possible to reduce, inhibit or even prevent zinc uptake by the patient's muscles by providing a metal-ion transporter, such as a zinc transporter, as a critical mediator, a therapeutic target, or a biomarker. A method for evaluating bioavailable zinc is known from U.S. Pat. No. 9,310,353. This can be used to determine when zinc levels are too high such that the metal-ion transporter would need to be administered.

The present invention preferably targets the chelator or inhibitor so that it is directed to the patient's muscle or at least to the vicinity of the patient's muscle. This can be achieved by targeting the muscle using the techniques disclosed in the following references: PCT application WO 2015/116568-A1, U.S. Pat. No. 9,415,018 or 9,486,409, European application EP 2 488 165, or US patent publication 2015/0313699 A1.

The present invention provides a method for diagnosing a development or a progression of cachexia in a patient by monitoring Zip14-mediated zinc accumulation in the patient's muscle including either monitoring one or more of the expression level of Zip14 (also known as Slc39a14), the loss of myosin heavy chain (MyHC), or the reduction of muscle-cell differentiation in the patient's muscle. The present invention also provides a method for monitoring the development or progression of cachexia in a patient using Zip14 as a biomarker, such as by detecting an increased-level of Zip14 protein or an increased-level of Zip14-mediated zinc accumulation in the patient's muscle. The present invention specifically provides a method for treating or suppressing cancer-induced cachexia in a patient to increase the survival of the patient by administering a pharmaceutical composition in an effective amount to reduce a Zip14-mediated zinc accumulation in the patient's muscle, including administering a zinc chelating agent, an inhibitor of Zip14 protein, or a nucleic acid to reduce or eliminate the expression of Zip14.

The diagnosing and treating methods of cachexia of the present invention are based on the surprising finding that Zip14, a zinc transporter, were characterized as a critical mediator in the development of metastasis-induced cachexia through perturbed zinc homeostasis by mediating zinc overload in skeletal muscle in promoting cancer-induced muscle atrophy. In addition, Zip14 also was characterized as a critical mediator for inducing myofibrillar protein loss and blocking new muscle regeneration.

Based on the surprising finding, the present invention discloses a method to treat or diagnose cachexia using a zinc transporter as a mediator, a therapeutic target, or a biomarker, wherein the cachexia is induced by cancer or other disorders, such as COPD (chronic obstructive pulmonary disease), AIDS (acquired immune deficiency syndrome), and renal diseases. In one embodiment, the zinc level in patient's muscle is controlled by restricting zinc uptakes in patient's diet, such as providing zinc-free water, zinc-free food or combinations thereof. In addition, the zinc level in muscle of the patient is controlled by administering a zinc chelating agent, a zinc transporter inhibitor which can inhibit the function of the zinc transporter protein, or a nucleic acid which can reduce or eliminate the expression of the zinc transporter gene. Furthermore, the present invention provides a method to diagnose the susceptibility or progression of cachexia using Zip14 as a biomarker, wherein the method comprises monitoring an expression profiling of Zip14 gene and a zinc level in muscle of the patient.

In one embodiment, a Zip14 was significantly upregulated in the cachectic muscles from metastasis models and was expressed specifically in the atrophic muscle fibers from advanced cancer patients. Zip14 promoted muscle mass loss and blocked muscle regeneration in cancer as shown in the obtained results using Zip 14-null mice and in vivo muscle-specific Zip14 knockdown. It demonstrated that Zip14-mediated zinc influx in muscle cells was critical for the development of metastasis-induced cachexia.

In one embodiment, upregulated Zip14 expression was observed in cachectic muscles from mice and patients with metastatic cancer and was required for aberrant accumulation of zinc in muscle. Zip14-mediated zinc uptake in muscle progenitor cells caused the repression of the key myogenic factors, MyoD and Mef2c, and reduced muscle-cell differentiation. Zip14-mediated zinc accumulation in differentiated muscle cells induced the loss of myosin heavy chain protein. Surprisingly, metastasis-induced cachexia was reduced by the knockdown, knock-out or targeted depletion of Zip14.

One of the common characteristics of cancer cachexia is a shift towards protein catabolism through activation of the ubiquitin-mediated proteasome degradation system and autophagy pathways. The present invention demonstrates that Zip14-mediated zinc accumulation in muscle cells leads to the loss of myosin heavy chain (MyHC) protein expression. MyHC loss in muscles has been observed in cancer cachexia patients and in a variety of animal models suggesting that this typically abundant myofibrillar protein greatly impacts muscle size and function. In one embodiment, excess zinc uptake by myoblasts represses the myogenic transcription factors, MyoD and Mef2c, which may lead to blocking muscle-cell differentiation. Since these processes contribute to muscle atrophy in metastatic cancers, monitoring zinc consumption in metastatic cancer patients using Zip14 as a biomarker or a therapeutic target can provide a method to diagnose the development of cachexia or to provide treatment for cachexia.

### EXAMPLE

The following examples illustrate the benefits and advantages of the present invention.

### Methods

Cell culture: KP1, C26 (parental), 4T1, and PC9-BrM3 cells were obtained from Stanford University, NCI-Frederick DCI Tumor depository, Princeton University and Memorial Sloan Kettering Cancer Center, respectively. C26m2 cells were derived from C26 parental cells by in vivo selection. C26 parental cells were purchased from NCI (National Cancer Institute). Human primary skeletal myoblasts were purchased from Lonza. C2C12 and 293T were purchased from ATCC. C26, C26m2, 4T1, and PC9-BrM3 cells were cultured in RPMI (purchased from Life Technologies) containing 10% FBS (purchased from Sigma). KP1 cells were cultured in RPMI containing iron supplemented 10% bovine growth serum (purchased from Hyclone). 293T and C2C12 cells were cultured in DMEM (purchased from Life Technologies) containing 10% FBS. Mouse primary myoblasts were cultured in Hams F-10 (purchased from Life Technologies) containing 20% FBS and 2.5 ng/ml of bFGF. All the media were supplemented with 1×Pen/Strep (100 lU/ml of Penicillin and 100 pg/ml of Streptomycin from Life Technologies). Human primary skeletal myoblasts were cultured in SKGM-2 Bullet kit media (purchased from Lonza).

Adenoviral infection: C2C12 cells or mouse primary myoblasts were cultured overnight. C2C12 cells were infected with adenovirus expressing either GFP (green fluorescent protein) control (Adeno-GFP) or mouse Zip14 (Adeno-Zip14, purchased from Vector Biolabs). Primary myoblasts were infected with adenovirus expressing either GFP control (Adeno-GFP) or mouse Zip14 (Adeno-Zip14).

Muscle differentiation assays: Differentiation was initiated after adenoviral infection by switching the growth medium to differentiation medium (DMEM containing 2% horse serum and 5 μg/ml of insulin for C2C12 cells; DMEM containing 2% horse serum without insulin for primary myoblasts) the day after infection. Differentiation medium was changed at designated time-points.

Zinc and MG132 treatment of muscle cells: 3-day differentiated C2C12 cells were cultured with 50 μM ZnCI₂ in differentiation medium for 24 hours. Cells were then used for immunofluorescence staining, gene expression analysis and immunoblot analysis. For MG-132 (benzyloxycarbonylleucyl-leucyl-leucine aldehyde, a proteasome inhibitor) treatment, 3-day differentiated C2C12 cells expressing Zip14 (Adeno-Zip14) were treated with 50 μM ZnCI₂ for 24 hours, and then treated with either vehicle (DMSO) or MG132 (50 μM) for 3 hours prior to harvest for immunoblot analysis.

Cell viability assay: Viability of C2C12 cells was determined by MTS assay (a cell proliferation assay based on a colorimetric method for quantification of viable cells in proliferation and cytotoxicity assay) using CellTiter 96® Aqueous One Solution Cell Proliferation Assay kit (purchased from Promega) containing tetrazolium compound. C2C12 cells infected with Adeno-GFP control or Adeno-Zip14 were plated in growth media and differentiated. Cells were treated with 50 μM of ZnCl₂ for 24 h. Cell viability was measured by adding 100 μl of growth medium without phenol red to each well after aspirating media from the wells. 20 μl of CellTiter 96 AGueous One Solution Reagent was added to each well. After 1 hour of incubation at 37° C. in CO₂ incubator, the amount of soluble formazan was determined by absorbance at 450 nm. Undifferentiated and differentiated C2C12 cells were collected for immunoblot analysis probing for cleaved-caspase-3 expression to assess cell-death. C2C12 cells treated with the indicated doses of doxorubicin (doxo) (purchased from Sigma) served as positive control for both types of viability assays.

Treatment with cytokines and signaling pathway inhibitors: Murine C2C12 myoblasts and human primary skeletal myoblasts were serum-starved overnight, and then treated with or without inhibitors of the TGFβ/Smad, NFκB and c-jun/AP1 pathways, which are SB431542 (purchased from Thermo Fisher), CC401 (purchased from Thermo Fisher) and BAY 11-7085 (purchased from Enzo), respectively, followed by treatment with recombinant cytokines purchased from R&D Systems (recombinant mouse TNFα and TGFβ1 at 50 ng/ml and 10 ng/ml, respectively, for C2C12, recombinant human TNFα and TGFβ1 at 50 ng/ml and 10 ng/ml, respectively, for human primary skeletal myoblasts). Cells were pretreated with either vehicle (DMSO) control, or 10 μM of the respective pathway inhibitors for 1 hour, and then treated with TGFβ1 for 9 hours or TNFα for 3 hours before harvest. Cells with different treatments were harvested together for subsequent analysis.

Zinc uptake assay: Control or Zip14-expressing C2C12 cells were cultured and washed with serum-free and phenol red-free DMEM. Cells were then incubated with DMEM containing 0.5 μM of ZnCI₂ in 5% CO₂ cell culture incubator at 37° C. ZnCI₂ levels remaining in the culture medium at 0, 1, 2, and 3 hours were determined with FluoZin-3 (purchased from Thermo Fisher), a zinc-specific fluorescent chelator. Specifically, 10 μl of medium was taken out from the plate at the designated time points and mixed with 90 μl of FluoZin-3 in PBS to give final FluoZin-3 of 3 μM. The mixture was incubated for 5 minutes at room temperature in the dark, and fluorescence was detected by a plate reader. The linear standard curve of fluorescence signal was determined by ZnCI₂ with known concentrations between 0 to 10 μM.

Generation and validation of antibodies against human and mouse Zip14: Codon optimized synthetic cDNA fragment (g-Block purchased from IDT) encoding soluble cytoplasmic domain of human (amino acids 246-352) and mouse (amino acids 243-349) Zip14 were used for the generation of antibodies. Polyclonal antibodies against both purified human and mouse Zip14 domains were produced in rabbits. To validate antibodies, Western blot was performed on crude bacterial lysates (uninduced and induced) using the immunized sera. Immunized sera against human or mouse Zip14 only detected Zip14 domains in the induced crude lysates, confirming antibody specificity. The specificity of the purified IgGs were validated by immunohistochemical analyses using liver sections from Zip14 know-out (KO) mice (negative control) and from Zip14 wild type (WT) mice (positive control).

Immunohistochemical staining: Paraffin-embedded tissues were sectioned at 5 μm thickness. Slides were baked at 60° C. for 1 hour and de-paraffinized, rehydrated, and treated with 1% hydrogen peroxide for 10 mins (except for TGFβ staining, which was treated with 0.6% hydrogen peroxide in methanol for 1 hour. Antigen retrieval was performed in citrate buffer (pH 6.0) in a steamer with the exception of TGFβ immunostaining, in which 1 mg/ml of hyaluronidase in 0.1 M of sodium acetate buffer (pH 5.5) was used for 30 mins digestion at 37° C. Endogenous avidin/biotin were blocked, and for TGFβ, endogenous mouse IgG was also blocked. After the slides were further blocked with 3% BSA in PBS containing 10% goat serum, tissue sections were incubated with primary antibody including rabbit polyclonal antibodies against Zip14 (1:250 of 06-1022 from Millipore, and 1:1000 of HPA016508 from Sigma), rabbit polyclonal antibodies against human Zip14 (1:500) or mouse Zip14 (1:2500) developed in Columbia University, rabbit polyclonal antibody against TNFα (1:100 of 210-401-321 from Rockland), and mouse monoclonal antibody against TGFβ (15 μg/ml of clone 1D11.16.8 from BioXCell), followed by corresponding biotinylated secondary antibodies. ABC kit and DAB kit (Vector laboratories) were used for detection. Sections were subsequently counterstained with Hematoxylin, dehydrated and mounted using Cryoseal XYL (Richard-Allan Scientific) for subsequent histological analysis.

Mice and genotyping: Balb/c and C57BI/6 mice were obtained from Jackson Laboratories. DBA/2 and 129P2/Ola mice were obtained from Envigo. Zip14 knockout (KO) mice generated by Hojyo and Fukada laboratory and were obtained on a congenic Balb/c background from the Knutson Laboratory (University of Florida). C57BI/6 were crossed with 129P2/Ola to generate 129P2/Ola×C57BI/6 mice; Balb/c were crossed with DBA/2 to generate CD2F1 mice, and Zip14 mice were crossed with DBA/2 to generate Zip14 knockout mice in CD2F1 background. K-ras^{LSL-G12D/+}, p53^{fl/fl} and Lkb1^{fl/fl} mice were obtained from the NCI Mouse Repository. K-ras^{LSL-G12D/+} were crossed with p53^{fl/fl} to generate K-ras^{LSL-G12D/+}-p53^{fl/fl} mice, and K-ras^{LSL-G12D/+} were crossed with Lkb1^{fl/fl} to generate K-ras^{LSL-G12D/+}-Lkb1^{fl/fl}.

Metastasis assays in mice: Both male and female mice were used in this study. Athymic mice aged 8-9 weeks were injected with 1×10⁵PC9-BrM3 cells by intracardiac route into arterial circulation for experimental metastasis assays. For C26m2, 4T1 and KP1 tumor studies, mice aged between 5-6 weeks for C26m2, 8-9 weeks for 4T1 and 4-5 weeks for KP1 injections were used. For each model, 1×10⁶ tumor cells were subcutaneously injected in the right flank of syngeneic mice. Subcutaneous tumor was removed between 2-3 weeks to allow for metastasis formation following the tumor-resection-relapse approach. Zip14 WT or Zip14 KO mice in CD2F1 or Balb/c background at 4-5 weeks of age were subcutaneously injected with 1×10⁶C26m2 or 4T1 tumor cells, respectively. Tumors were not resected with survival-surgeries in the Zip14 WT and KO mice due to the phenotypic and behavioral abnormalities in the Zip14 KO mice. Instead, spontaneous metastasis in the presence of tumors was monitored by bioluminescent imaging at endpoint of 5 weeks post-tumor cell injection in the Zip14 WT and Zip14 KO groups.

Neutralization assay of TNFα and TGFβ in mice: Athymic and Balb/c mice of 8-9 weeks of age were subcutaneously injected with C26m2 and 4T1 cells, respectively. Samples/mice were recorded by randomized cage numbers generated on Filemaker pro and treatment groups were assigned based on those numbers. The primary tumor were surgically removed 2-3 weeks after tumor cell injection. One week after tumor removal, lnVivoPlus anti-TGFβ (BP0057, Clone: 1D11.16.8), lnVivoPlus anti-TNFα (BP0058, Clone: XT3.11) or lnVivoPlus Mouse IgG 1 Isotype control (BP0083, Clone: MOPC-21) from BioXCell were intraperitonealy injected into mice with a dose of 200 μg/mouse three times a week for 10 days.

Zinc-supplemented water treatment for mice: ZnS0₄ solution was purchased from Sigma. Zip14 WT and KO mice were given either regular water or zinc-supplemented drinking water (25 mM ZnS0₄ in their drinking water). Zinc water was started from the day of tumor injection in the tumor-bearing group and in matched uninjected controls, which continued until the animals were euthanized at 15 days. Tumors were not resected because cachectic symptoms started to develop early and were visible between 8-10 days in the tumor-bearing Zip14 WT group of mice on zinc-enriched water.

Behavioral coordination tests in mice: Rotamex-5 (Columbus Instruments) with a rod diameter of 3 cm, was used for testing coordination in mice. In this setup, automatic fall detection is implemented within each lane by a series of photocells placed above the rotating rod. The speed of the rotating rod is programmed for either constant or accelerated modes. Rod speed can be specified in either terms of rotations (RPM) or in linear terms (cm per second). Latency to fall is detected with 0.1 second temporal resolution. Rate of rotation at time of fall is resolved to 0.1 RPM or 0.1 cm/second. Both latency and rod speed at time of fall are presented on a display for each of the four lanes. When operated in accelerating mode, Rotamex-5 allows entry of acceleration increment and interval over which the acceleration should occur. For each mouse, an average of 3 runs are recorded, with 5 minutes rest between each run. The speed that the rod is spinning at when the mouse falls is measured in RPMs. The time it takes for the mouse to fall is measured in seconds. The mice are placed on the rod for 1 minute while the rod spins at 1 RPM so the mouse gets used to the rod spinning. When the experiment begins the rod accelerates at 1 rpm every 10 sec until the mouse falls off. When the mice fall off the speed of the rod and amount of time the mouse was on the rod are recorded.

Virus production, purification and titration: For adeno-associated virus production, two different AAV vectors were constructed, including AAV-CAG-Zip14-IRES-GFP and AAV-CAG-mCherry. pAAV-Ef1a-mCherry-IRES-Cre (Addgene plasmid #55632) was a gift from Karl Deisseroth, and was used as PCR template for cloning mCherry and IRES. AAV-CAG-ChR2-GFP (Addgene plasmid #26929) was a gift from Edward Boyden, and was used as template for cloning GFP. AAV-CAG-ChR2-GFP was also used as backbone AAV vector with CAG promoter after digesting by BamHI (Roche) and BsrGI (Thermo Fisher). By sequential PCR amplification, mCherry alone, Zip14-IRES-GFP amplicons, containing N-terminal BamHI and C-terminal BsrGI digestion sites, were introduced into digested AAV-CAG backbone to get the AAV vectors. The AAV constructs were confirmed by sequencing, and then co-transfected with pDeltaF6 and AAV 2/9 Helper plasmids, in a ratio of 1:2:1.6, into 293T cells by calcium phosphate. 48 hours later, 293T cells containing AAV were collected for virus purification.

To purify viruses, AAV9-producing 293T cells were detached by adding 1/80 volume of 0.5 M EDTA (pH 8.0) for 10 mins incubation at room temperature and collected by centrifugation at 2,000×g for 10 mins at 4° C. Cell pellets were lysed by adding 24 ml of 0.5% Triton X-100 in PBS containing 5 μg/ml of RNase A (Sigma) and shaking for 1 h at 37° C. Cell lysates were centrifuged at 10,000×g for 10 mins at 4° C., and 24 ml of supernatant was added into an ultracentrifuge tube. The virus solution was raised up by successive addition of 3 ml of 25% iodixanol, 4 ml of 40% iodixanol and 2 ml of 60% iodixanol to the bottom of the tube. All the iodixanol solutions were prepared in PBS containing 1M NaCI, 1 mM MgCl₂, 2.5 mM KCl. The tube was centrifuged at 350,000×g for 1.5 hours at 18° C. 4.5 ml of virus solution at the bottom of tube was collected using 18 G needle and filtered through 0.45 pm filter. Virus solution was then concentrated using Amicon Ultra-15 (100K) (Millipore) and washed 3 times with 250 mM NaCl solution. Virus titration was performed with primers targeting at CAG (forward 5′-TTA CGG TAA ACT GCC CAC TTG-3′, reverse 5′-CAT AAG GTC ATG TAC TGG GCA TAA-3′) with AAV-CAG-mCherry plasmid as standard.

AAV9 injection: AAV9-mCherry-U6-mSLC39A14-shRNA or AAV9-mCherry-U6-scrmb-shRNA (both were purchased from Vector Biolabs) was used for knockdown of Zip14 expression or as negative control, respectively, through injection to mouse muscles. The validated shRNA sequence for knockdown of Zip14 is CCGGGCAGGCTCTCTTCTTCAACTTCTCGCGAAGTTGAAGAAGAAGAGAGCCTGC-TTTTTG (purchased from Vector Biolabs). Zip14 knockdown efficiency was confirmed to be about 90% in Hepa1.6 cells (purchased from Vector Biolabs). For knockdown of Zip14 in mouse skeletal muscle, 3×10¹¹ genome copies of AAV9 virus were injected into the right gastrocnemius muscle using five injection sites in 7-8 weeks old athymic mice. mCherry expression was monitored weekly by fluorescence imaging, and after confirmation, tumor growth and metastasis assays were performed. Athymic mice were used to avoid additional immune reaction. For overexpression of Zip14, 2.2×10¹⁰ genome copies of AAV9 virus purified above were injected into the gastrocnemius muscle using five injection sites in 5-6 weeks aged Zip14 KO Balb/c mice.

Single myofiber isolation and LA-ICP-MS: Single myofiber isolation from EDL (extensor digitorum longus) muscles was performed. EDL muscles were dissected and transferred into a prewarmed horse serum coated Petri dish containing 1.8 ml of DMEM supplemented with 10% FBS, 1×pen/strep antibiotics and 110 mg/ml of sodium pyruvate. Then 0.2 ml of 2% collagenase (about 40,000 U/ml) solution was added and muscles were digested at 37° C. in a 5% C0₂ incubator for 40 to 60 mins, during which a large bore glass pipette for flushing the muscle would help to loosen up the muscle and release single fibers into medium. The released muscle myofibers were transferred into a pre-warmed horse serum coated Petri dish with 4 ml of DMEM containing 10% FBS and 110 mg sodium pyruvate to avoid over-digestion. The myofibers were then transferred into a pre-warmed horse serum coated Petri dish containing wash media (DMEM supplemented with 1×pen/strep and 110 mg/ml of sodium pyruvate), and washed for three times to remove dead myofibers and debris. Single myofibers were transferred onto glass slide and air-dried. For subsequent LA-ICP-MS (Laser Ablation Inductively Coupled Plasma Mass Spectrometry) analysis, single muscle fiber mounted on slide were placed in sealed ablation cell and ablated with a new wave UP213 Nd:YAG laser beam at 0.25-0.35 mJ with a 100 pm spot size. Ablation was set at 5 pm/sec and 20 Hz. The ablated sample particles were then transferred to a Thermo iCapQ ICP-MS that was optimized using a NIST 612 glass standard prior to every sample run. The isotopes selected for analysis were ⁶⁴Zn, ⁶⁶Zn and ³¹P. Individual muscle fiber data was subtracted from a blank line on the same slide with same dimension, size and parameters as the sample line.

Liver and kidney function analysis: Liver and kidney function tests were performed using automated clinical chemistry analyzer (VetAce® Clinical Chemistry System; Alfa Wasserman Diagnostic LLC West Caldwell, N.J.) for AST (aspartate aminotransferase), BUN (blood urea nitrogen) and creatinine in serum. Specifically, 20 pi, 3 pi and 20 pi of serum from mice bearing C26m2 metastases with or without zinc supplemented water treatment were used for assays of AST, BUN and creatinine, respectively.

Magnetic sorting of muscle satellite cells: CD45⁻CD31⁻Sca1⁻lntegrin-α7⁺ skeletal muscle satellite cells were isolated according to the methods described. All limb skeletal muscles from 1-2 week old mice were combined and minced into a smooth pulp. The muscles were then digested with collagenase (2-5 ml of 0.2% collagenase type 2, based upon muscle mass, in DMEM with 10% FBS) at 37° C. for 40 mins. The dissociated single cells were filtered through 70-micron strainer and pelleted by centrifugation at 400×g for 5 mins at 4° C. Cells were washed twice with DMEM containing 2% FBS and suspended in 200-500 μl of DMEM with 2% FBS. Fc blocker (1:100, BD Pharmingen, 553142) was added to the cell suspension and incubated on ice for 10 mins. The following antibodies were then added into the cell suspension: CD31-PE (1:100, eBioscience, 12-0311-81), CD45-PE (1:100, eBioscience, 12-0451-83), Sca1-PE (1:100, eBioscience, 12-5981-81), integrin-α7 antibody (1:10, Miltenyi Biotec, 130-103-774), and the mixture was shaken at 4° C. for 15 mins. Cell pellet was washed twice with DMEM containing 2% FBS, and resuspended in DMEM with 2% FBS. 40-100 μl of anti-PE magnetic beads (Miltenyi Biotec, 130-105-639) was added into the cell suspension and the mixture was shaken at 4° C. for 15 mins. Cell pellet was washed twice with MACS buffer (PBS with 0.5% BSA and 2 mM EDTA), resuspended with 0.5 ml of MACS buffer, and applied onto a LD column that was set up on a magnetic board (Miltenyi Biotech). The flow-through cells were collected, and pelleted by centrifugation. The cells were then resuspended with 80-200 μl of DMEM with 2% FBS, and 20-50 μl of anti-mouse IgG magnetic beads (Miltenyi Biotec, 130-048-402) was added into the cell suspension. The mixture was shaken at 4° C. for 15 mins, and the cell pellet was washed twice with MACS buffer. Cells were then resuspended with 0.5 ml of MACS buffer, and applied onto an LS column. After washing with MACS buffer, the cells retained in the LS column were collected. Isolated muscle satellite cells were cultured in collagen-coated dishes with myoblast growth medium.

Isolation of muscle progenitor cells by flow cytometry: Mouse gastrocnemius muscles were collected and processed for depletion of CD45⁺ and CD31⁺ cells by anti-PE magnetic beads using LD column (Miltenyi Biotech) under Magnetic Sorting of Muscle Satellite Cells. Cells in the flow-through fraction were pelleted by centrifugation and resuspended with 100 pi of DMEM containing 2% FBS. The following antibodies were then added into the cell suspension: CD34-FITC (1:50, Miltenyi Biotec, 130-105-831), Sca1-PE (1:100), and integrin-α7-APC (1:100, Miltenyi Biotec, 130-103-356). The mixture was shaken at 4° C. for 45 mins in dark, and then washed twice with FACS buffer (0.5% BSA in PBS). The cells were resuspended in FACS buffer and used for flow cytometric analysis for isolation of CD34⁺Sca1⁺ and CD34⁺integrin-α7⁺ cells.

Subcellular fractionation of differentiated C2C12 muscle cells: Fractionation of soluble and myofibrillar components was performed. Differentiated C2C12 muscle cells were collected in cold lysis buffer (20 mM of Tris-HCl pH 7.2, 5 mM of EGTA, 100 mM of KCl, 1% Triton X-100, and 1× protease and phosphatase inhibitor cocktail), and lysed by gentle agitation at 4° C. for 1 h. After centrifugation at 3,000×g for 30 mins at 4° C., the cytosolic fraction (supernatant) was collected and stored in −80° C. The pellet (myofibrils) was washed twice with wash buffer (20 mM of Tris-HCl, pH 7.2, 100 mM of KCl, and 1 mM of DTT). After centrifugation at 3,000×g for 10 mins at 4° C., myofibrillar fraction was extracted in ice-cold extraction buffer (0.6 M of KCl, 1% Triton X-100, 2 mM of EDTA, 1 mM of DTT and 1× protease and phosphatase inhibitor cocktail) with shaking at 4° C. The purified myofibrillar fraction was collected after centrifugation for 3,000×g at 4° C. and stored in −80° C. until further use.

Succinate dehydrogenase (SDH) staining of mouse muscles: Cryosections of mouse muscles were incubated with 1 mg/ml of nitrotetrazolium blue chloride and 100 mM of sodium succinate in PBS at 37° C. for 30 mins. Slides were washed three times with PBS and mounted with glycerol.

Statistical analysis: Statistical significance was determined by unpaired two-tailed Student's t-test, Welch's t test, Pearson Chi-Square test or One-way ANOVA with post-hoc Tukey's test using Prism 6 software (GraphPad Software) as indicated in the figures and legends. All values are mean±SEM and p-value <0.05 was considered statistically significant.

### Example 1. Development of Metastasis-Induced Cachexia Models

Allografts were performed using 4T1 and C26m2 cells to develop metastasis-induced cachexia models to investigate the mechanisms of developing muscle wasting during the advanced stages of cancer. 4T1 cell was a well-established murine model of breast cancer metastasis. C26m2 cell was a metastatic subline of C26 murine colon cancer cells that were generated by in vivo selection approach.

The tumor-resection-and-relapse approach (Francia, G., Cruz-Munoz, W., Man, S., Xu, P. & Kerbel, R. S. Mouse models of advanced spontaneous metastasis for experimental therapeutics. Nature reviews. Cancer 11, 135-141, 2011) was used for spontaneous metastasis development using C26m2 and 4T1 cells to induce cachexia during metastatic progression. Both cell lines were engineered to express luciferase and implanted subcutaneously respectively. Resulting tumors were resected two to three weeks later after confirming with bioluminescence imaging that there was no detectable signal at the implanted site. Two to three weeks following tumor removal, distant metastases and a concomitant reduction in body weight and grip strength were detected in C26m2- and 4T1-implanted mice. Morphometric analysis of tibialis anterior muscle sections revealed that fiber diameters were markedly reduced compared to control muscles from non-tumor-bearing mice.

Marker genes of muscle atrophy (MuRF1, MAFbx/Fbxo32, Fbxo31, and Musa1/Fbxo30) that encode ubiquitin ligases were transcriptionally upregulated in the cachectic tibialis anterior and diaphragm muscles from the 4T1 and C26m2 metastasis models. The following muscle groups also showed induction of the muscle atrophy genes: 1) extensor digitorum longus (EDL) muscles with a predominance of fast-twitch, glycolytic fibers, 2) soleus muscles with a predominance of slow-twitch, oxidative fibers, 3) gastrocnemius and quadriceps with mixed-fiber types, and 4) cardiac muscles. Cachectic symptoms were not due to anorexia in either model. These results showed that cachexia in muscle groups of diverse fiber types were systemically induced by metastatic C26m2 and 4T1 cancer cells, which is similar to human cancers. These metastatic models provide the advantages of eliminating the physical complications of large tumor burden for testing potential anti-cachexia treatments.

Mouse images and body weight analysis of tumor-bearing mice (Tb) and non-tumor-bearing control (Con) mice were analyzed. Using the tumor-resection-and-relapse approach for spontaneous metastasis assay, luciferase-labeled 4T1 or C26m2 cancer cells were implanted subcutaneously and after 2-3 weeks of tumor growth, tumors were surgically removed. Metastasis was monitored by bioluminescence imaging. Mice were euthanized with cachectic symptoms such as a body condition score <1.5, reduced body weight and hunched posture. n=9 for Tb (4T1), n=6 for Balb/c (Con); n=9 for Tb (C26m2), n=10 for CD2F1 (Con). Hind-limb grip strength measurements of mice bearing 4T1 or C26m2 metastases at 5 weeks post tumor-cell injection (n=5 per group) were conducted. Immunofluorescence images and associated morphometric analysis of cross-sections from tibialis anterior (TA) muscles harvested from mice at 5 weeks post tumor-cell injection were compared to their respective non-tumor-bearing controls (n=4 mice per group). Sections were immunostained with antibody against laminin and stained with DAPI. Morphometric analysis was conducted as the distribution frequency of fiber size categorized by fiber diameter. Quantitative RT-PCR (qRT-PCR) analysis of muscle atrophy markers MuRF1, MAFbx/Fbxo32, Fbxo31, Musa1/Fbxo30 in TA and diaphragm muscles were conducted. For TA muscles, n=4-6 controls and n=6-7 mice using the 4T1 model; n=5 controls and n=3-5 mice using the C26m2 model. For diaphragm muscles, n=6 mice per group for both 4T1 and C26m2 models. The in vivo selection process for C26m2 cell line derivation was conducted. Luciferase-labeled murine colon cancer C26 parental (C26p) cells were injected into CD2F1 mice via intracardiac injection into the arterial circulation to generate liver metastases. Cancer cells were isolated from the liver and purified by antibiotic selection. Cells were passaged in syngeneic mice for another round of selection, and the resulting C26m2 metastatic cell line was subsequently used for cachexia studies. Mice were subsequently monitored for the development of cachexia and metastasis by bioluminescence imaging at 5 weeks post tumor-cell injection. qRT-PCR analysis was conducted for MuRF1, MAFbx/Fbxo32, Fbxo31 and Musa1/Fbxo30 expression in the gastrocnemius, quadriceps, soleus, EDL and cardiac muscles from mice bearing tumors (Tb) derived from either 4T1 (f) or C26m2 (g) metastases. Muscles were collected for analysis 5 weeks after tumor cell injection and were compared with the age-matched, non-tumor-bearing controls (Con).

### Example 2. Gene-Expression Analysis

To determine the mechanisms mediating the development of cachexia in the C26m2 and 4T1 metastatic models, the transcriptome of the cachectic tibialis anteriormuscles of both models were analyzed by RNA sequencing. Unsupervised principal component analysis showed that gene expression profiles from cachectic muscles segregated independently from their respective controls. Significantly concordant transcriptional changes in the C26m2 and 4T1 models with 3140 common differentially expressed genes were observed. The results indicated overlapping mechanisms. Functional annotation clustering of the common genes using DAVID (Database for Annotation, Visualization and Integrated Discovery) identified 5 clusters with upregulated genes and 4 clusters with downregulated genes with enrichment scores (ES) >5.0 (p<0.05).

A marked enrichment in pathways associated with protein degradation (autophagy and proteasome) was observed in cachectic muscles by the following three independent analyses: 1) functional annotation clustering using DAVID, 2) Gene Set Enrichment Analysis (GSEA) using KEGG pathway gene sets, and 3) quantitative RT-PCR for genes associated with ubiquitination, ubiquitin-proteasome and autophagy-lysosomal systems. Unexpectedly, genes associated with zinc binding and zinc transport were significantly enriched in the cachectic muscles from the 4T1 and C26m2 metastasis models (ES=12.08, p<0.00001). In particular, the zinc transporter Slc39a14 (also known as Zip14) was highly upregulated in the cachectic tibialis and diaphragm muscles and uniquely upregulated among multiple zinc transporters. Zip14 upregulation was also observed in the cachectic gastrocnemius, quadriceps, soleus, EDL and cardiac muscles, indicative of Zip14 upregulation in multiple muscle groups during cachexia development.

Transcriptomic profiling was conducted by RNA-Seq analysis of tibialis anterior (TA) muscles collected from mice with 4T1 or C26m2 metastases (Tb) or non-tumor-bearing, age-matched controls (Con) at five weeks post tumor-cell injection. The full list of differentially expressed genes common between the two models with significant p values and q values (cutoff=0.05) was sorted by decreasing log 2 fold change in C26m2 data. Functionally annotated clusters were determined by DAVID analysis using the common differentially expressed genes between the 4T1 and C26m2 models, with a cutoff of log 2 fold change of 1.0 and significant p and q values. Significant functional clusters of upregulated genes with an enrichment score (ES) >5.0 and p value <0.05. n=2 mice per group. Top 10 commonly upregulated genes in cachectic muscles from 4T1 and C26m2 metastasis models was sorted by decreasing log 2 fold change (in C26m2 data) with g-value cutoff of 0.05 were identified in heatmap. Expression levels of the Slc39 family of zinc influx transporter genes in cachectic muscles from RNA-Seq analysis are relative to their respective controls. Slc39a14 (Zrt- and Irt-like protein 14, also known as Zip14) appeared on the heatmaps.

### Example 3. Zip14 Expression During Metastasis-Induced Cachexia

To explore whether Zip14 upregulation is a common phenomenon during metastasis-induced cachexia, genetically-engineered mouse models (GEMMs), xenograft and allograft models of metastatic lung cancer were analyzed (Kwon, M. C. & Berns, A. Mouse models for lung cancer. Mol Oncol 7, 165-177, 2013). GEMMS of metastatic lung cancer driven by conditional expression of Kras^{G12D} combined with either p53 or Lkb1 deletion, and a xenograft model of EGFR-mutant PC9-BrM3 human lung cancer (Nguyen, D. X., et al. WNT/TCF signaling through LEF1 and HOXB9 mediates lung adenocarcinoma metastasis. Cell 138, 51-62, 2009), showed body weight loss and signs of muscle atrophy. Zip14 was also induced in the cachectic muscles of these models. To test whether Zip14 expression can be induced by metastasis in the absence of cachexia, an allograft mouse model of metastatic small cell lung cancer (SCLC) driven by conditional deletion of Rb and p53 that fails to induce cancer-associated muscle wasting was analyzed. The results indicated that upregulation of Zip14 in muscle is specifically associated with cachexia in metastatic models across several cancer types.

To evaluate the clinical relevance of Zip14 upregulation in human cachexia, immunohistochemical analyses using anti-Zip14 antibodies were performed on muscle sections from metastatic cancer patients. Blinded pathological examination revealed that 19 of 43 cancer patients with cachexia showed specific Zip14 staining in atrophic muscle fibers compared to 8 of 53 non-cachectic cancer patients (Pearson's Chi-square test, p=0.002). Zip14 staining was low in the non-atrophic fibers in muscles from both non-cachectic and cachectic cancer patients. Two additional anti-Zip14 antibodies were used to validate these findings. In conclusion, Zip14 protein was significantly elevated in the atrophic muscles of metastatic cancer patients with cachexia.

To identify soluble factors that can upregulate Zip14 during cachexia, the upstream signaling pathways in cachectic muscles were analyzed by Ingenuity Pathway Analysis (IPA). The list of differentially expressed genes were queried in cachectic muscles common to the 4T1 and C26m2 metastasis models for upstream transcriptional regulators. Candidate pathways were tested and found that treatment of murine C2C12 myoblasts and human primary muscle cells with recombinant TGFβ and TNFα proteins significantly induced Zip14 expression. Zip14 expression was blocked in both human primary muscle cells and murine C2C12 cells by 1) inhibition of TNFα-induced NF-κB activation with Bay11-7085 (but not by inhibition of TNFα-induced c-jun/AP1 activation with CC-401) and 2) inhibition of TGFβ-induced Smad phosphorylation with the TGF-βRI kinase inhibitor SB431542. TGFβ and TNFα cytokines are both intricately linked to cancer metastasis and cachexia and were readily detected in the C26m2 and 4T1 metastatic tumor microenvironments. Neutralization of TGFβ and TNFα cytokines using a pan-TGFβ neutralizing antibody (clone 1D11) or TNF-alpha neutralizing antibody (clone XT3.11) reduced Zip14 expression in the tibialis anterior muscles in 4T1 and C26m2 metastasis models. Consistently, Zip14 reduction was associated with a concomitant reduction in Smad2 phosphorylation and NF-κB activation. These findings suggest that TGFβ and TNFα cytokines contribute to Zip14 upregulation in cachectic muscles in metastatic cancer.

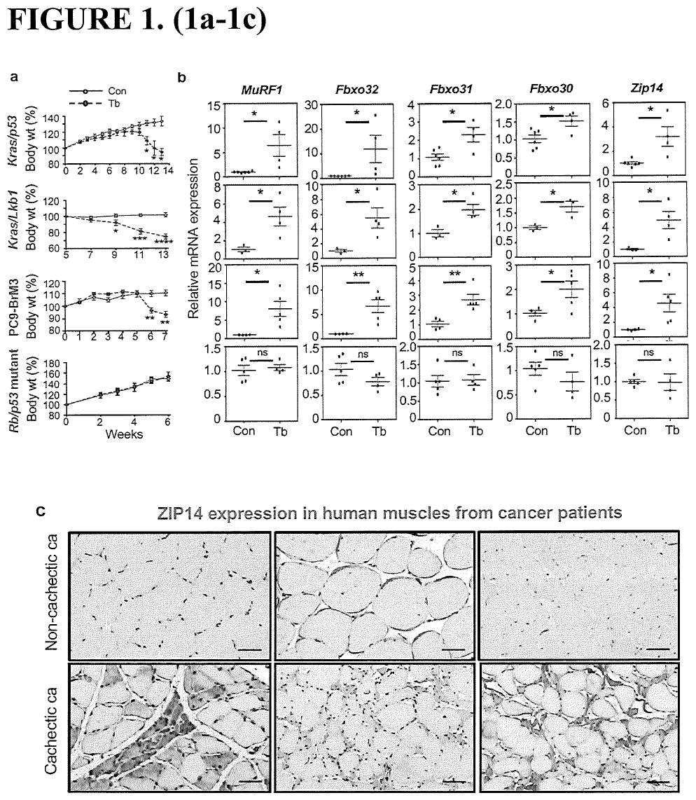

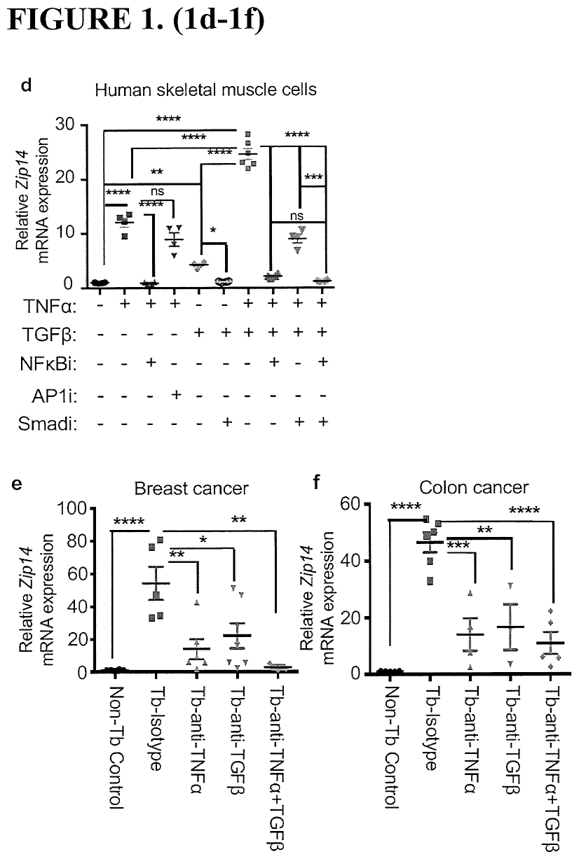

FIG. 1 shows the analysis results of Zip14 upregulation in cachectic muscles from multiple mouse models of metastasis-induced cachexia and from human cachectic cancer patients. FIGS. 1a and 1b: Body weight measurements (a) and relative qRT-PCR analysis of MuRF1, MAFbxlFbxo32, Fbxo31, Musa1/Fbxo30 and Zip14 in muscles (b) derived from four independent metastatic lung cancer models, compared to respective age-matched controls. Metastatic models include conditional Kras/p53 mutant (Kras^{LSL′G12D/+}-p53^{fl/fl}) and Kras/Lkb1 mutant (Kras^{LSL-G12D/+}-Lkb1^{fl/fl}) in which muscles were collected at 13 weeks post adeno-Cre induction, PC9-BrM3 xenograft in which muscles were collected at 7 weeks post tumor-cell injection, and Rb/p53 mutant allografts in which muscles were collected at 6 weeks post tumor resection. For body weight analysis in (a), n=4-8 for Kras/p53 mutant model, n=3-6 for Kras/Lkb1 mutant model, n=3-10 for PC9-BrM3 xenograft model, and n=3-10 for Rb/p53 mutant allograft model. For qRT-PCR analysis in (b), n=4-6 for Kras/p53 mutant model, n=3-4 for Kras/Lkb1 mutant model, n=4-5 for PC9-BrM3 xenograft model, and n=4-5 for Rb/p53 mutant allograft model. FIG. 1c: Representative images of Zip14 immunohistochemistry on human muscle cross-sections from non-cachectic (upper panel) and cachectic (lower panel) metastatic cancer patients. FIG. 1d: qRT-PCR analysis of Zip14 in human skeletal primary muscle cells treated with either vehicle, TNFα (50 ng/ml), TGFβ (10 ng/ml), or both TNFα (50 ng/ml)+TGFβ (10 ng/ml), either alone (vehicle) or in the presence of 10 μM of the indicated inhibitors. Cells were pretreated with either vehicle or the indicated inhibitors for 1 hour prior to adding the cytokines (TGFβ for 9 hours, and TNFα for 3 hours before harvest). Experiments were designed so that cells from all groups were harvested at the same time. n=4-6 samples per group. Inhibitors: NFκBi=NFκB inhibitor (BAY 11-7085); AP1i=AP1 inhibitor (CC401); Smadi=TGFβ receptor I inhibitor (SB431542).

FIGS. 1e and 1f: qRT-PCR analysis of Zip14 in TA muscles after neutralizing antibody treatment. Following the tumor-resection-and-relapse approach, mice injected with either 4T1 (e) or C26m2 tumor cells (f) were treated with either an isotype control antibody, or a neutralizing antibody against TNFa, TGFP, or both (200 pg antibody per mouse treated three times a week) starting one week after surgery, for a period of 10 days. n=3-8 mice per group for the 4T1 model, and n=3-6 mice per group for the C26m2 model.

Error bars represent SEM. p values were determined by two-tailed, unpaired Student's t test in (a, b) and with one-way ANOVA with post-hoc Tukey's test in (d-f). ns, not significant. *p<0.05, **p<0.01, ***p<0.001 and ****p<0.0001. Con, non-tumor bearing control; Tb, tumor-bearing.

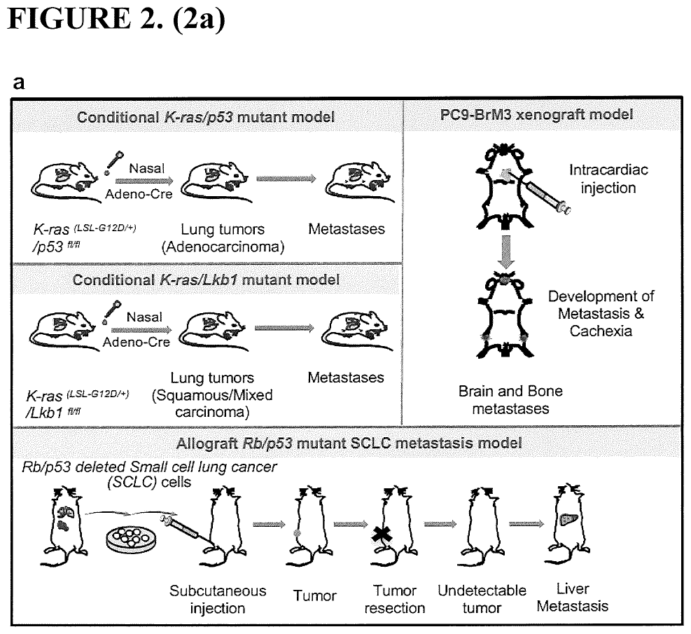

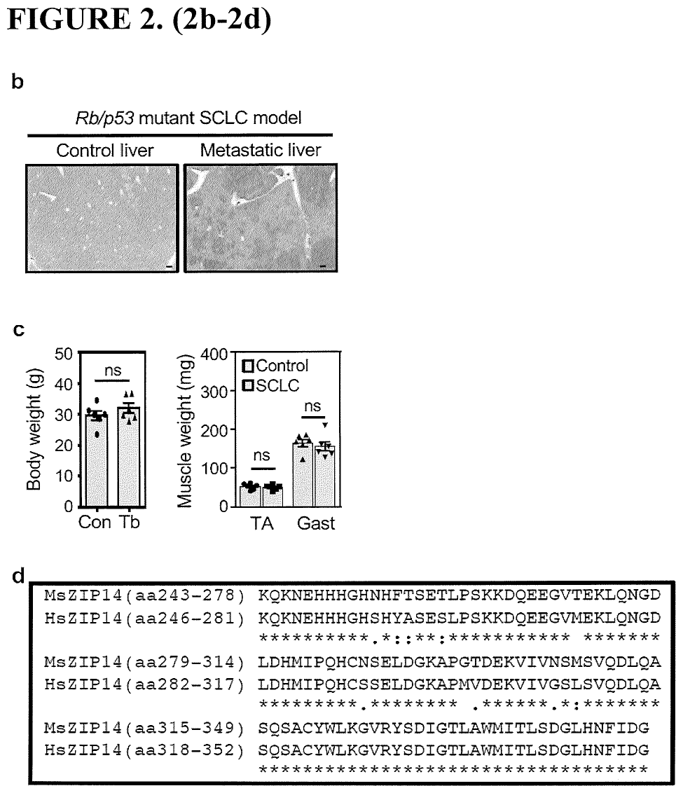

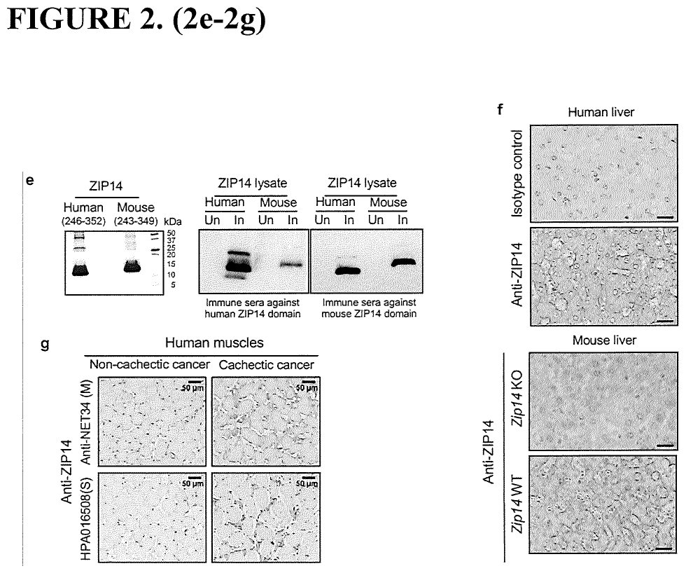

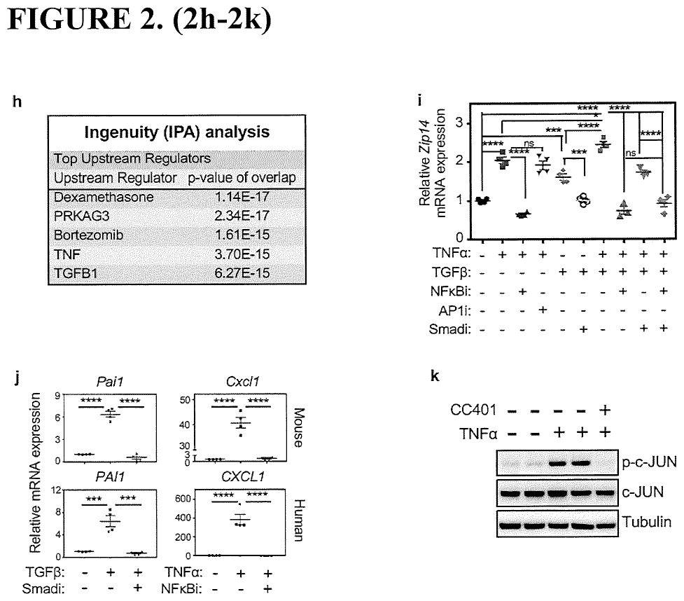

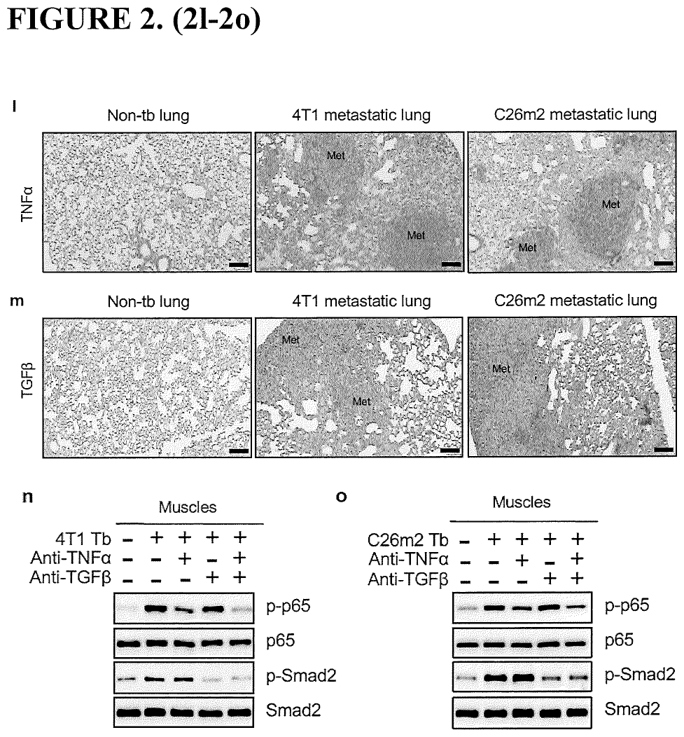

FIG. 2 shows tumor induction and metastatic progression in four metastatic models of lung cancer. FIG. 2a: Schematic representation of tumor induction and metastatic progression in four metastatic models of lung cancer. Autochthonous models Kras/p53 mutant (Kras^{LSL′G12D/+}-p53^{fl/fl}) and Kras/Lkb1 mutant (Kras^{LSL-G12D/+}-Lkb1^{fl/fl}), xenograft model PC9-BrM3, and Rb/p53 mutant (SCLC) allograft model were generated and characterized for cachexia development as shown.

FIGS. 2b and 2c: Representative images of H&E staining of liver sections (b) and body weight and limb muscle weights (c) from Rb/p53 mutant allograft model collected 6 weeks after tumor resection (Tb) and compared to age-matched, non-tumor-bearing control mice (Con). Scale bars, 100 pm in (b). n=6 mice per group in (c). Con, non-tumor-bearing control; Tb, tumor-bearing; TA, tibialis anterior; Gast, gastrocnemius muscle.

FIG. 2d: Amino-acid sequence of human (Hs) and mouse (Ms) Zip14 fragment used for generating antibodies against Zip14.

FIG. 2e: Images of Coomassie blue staining of purified recombinant human and mouse Zip14 protein fragment resolved on a 15% SDS-PAGE (left) and immunoblot analysis of uninduced (Un) and induced (In) crude bacterial lysates using immune sera against human and mouse Zip14 fragments (right).

FIG. 2f: Representative images of immunohistochemical staining of human liver (top two images) and mouse liver (bottom two images) using an antibody developed against human and mouse Zip14, respectively. Rabbit isotype antibody was used as a negative control for staining human liver sections, and Zip14 KO mice were used as a negative control for staining mouse liver sections. Scale bars, 25 pm.

FIG. 2g: Representative images of immunohistochemical staining using additional antibodies against Zip14 (commercial antibodies HPA16508 (S) and Anti-NET34 (M) generated using Zip14 peptide sequences). Scale bars, 50 pm.

FIG. 2h: Upstream regulators of Zip14 in cachectic muscles analyzed by Ingenuity Pathway Analysis (IPA). Differentially expressed genes common to the 4T1 and C26m2 models with p and q value <0.05 were used for querying upstream regulators. Pathways with significant p value <0.05 are listed with their p value of overlap.

FIG. 2i: qRT-PCR analysis of Zip14 in C2C12 myoblasts treated with either vehicle, TNFα (50 ng/ml), TGFβ (10 ng/ml), or both TNFα (50 ng/ml)+TGFβ (10 ng/ml), either alone or in the presence of the indicated inhibitors. Cells were pretreated with either vehicle or 10 μM of the indicated inhibitors for 1 hour prior to adding the cytokines (TGFβ for 9 hours, and TNFα for 3 hours before harvest). Experiments were designed so that cells from all groups were harvested at the same time. n=3-4 samples per group. Inhibitors: NFκBi=NFκB inhibitor (BAY 11-7085); AP1i=AP1 inhibitor (CC401); Smadi=TGFβ receptor I inhibitor (SB431542).

FIG. 2j: qRT-PCR analysis of Pail and Cxcll genes in mouse C2C12 cells and human skeletal myoblasts treated with TGFβ (10 ng/ml) alone or together with Smadi (TGFβ receptor inhibitor, SB431542), or TNFα (50 ng/ml) alone or together with NFκBi (NFκB inhibitor, BAY 11-7085). These genes were used as positive controls for testing the bioactivity of the recombinant TGFβ and TNFα proteins, and the efficacy of the inhibitors in pathway inhibition used in panel (i). n=4 samples per group.

FIG. 2k: Immunoblot analysis to detect abundance of phosphorylated c-JUN in 3 days differentiated C2C12 cells treated with either media control, 50 ng/ml of TNFα alone or 50 ng/ml of TNFα in the presence of 10 μM AP1 inhibitor (CC401) for 30 mins. Total c-JUN and tubulin were analyzed as loading controls.

FIGS. 2l and 2m: Representative immunostaining images of TNFα (l) and TGFβ (m) in lungs from either non-tumor-bearing mice or mice bearing 4T1 or C26m2 metastases at five weeks post tumor-cell injection. Scale bars, 100 pm.

FIGS. 2n and 2o: Immunoblot analysis of phosphorylated p65 and Smad2 in muscles after neutralizing TNFα or TGFβ antibody treatment. Following the tumor-resection-and-relapse approach, mice bearing either 4T1 (n) or C26m2 (o) metastases were treated with either an isotype control antibody, or a neutralizing antibody against TNFα, TGFβ, or both (200 μg antibody per mouse treated three times a week) starting one week after surgery, for a period of 10 days.

Error bars represent SEM. p values for (c) were determined by two-tailed, unpaired Student's t test, and p values for (i,j) were determined by one-way ANOVA. ns, not significant. *p<0.05, ***p<0.001 and ****p<0.0001.

### Example 4. Zip14 Upregulation and Zinc Accumulation in Muscle Mediates Cancer-Induced Cachexia

To determine whether Zip14 is required for the development of cancer-induced cachexia, cancer cells were implanted subcutaneously into Zip14 germline knockout and wild-type mice and evaluated the effects of Zip14 loss. Zip14 knockout mice are viable but display dwarfism, scoliosis, shortened bones, defective cartilage formation and behavioral problems. Upon tumor implantation, Zip14 knockout and wild-type mice developed metastasis and displayed similar tumor growth. Zip14-deficient mice were significantly resistant to cancer-induced muscle wasting. Examination of gastrocnemius, tibialis and EDL muscles revealed no change in the distribution of oxidative and glycolytic fibers, fiber-type switching, or vascularization between wild-type and Zip14-knockout mice in the presence or absence of tumor burden, as determined by succinate dehydrogenase (SDH) staining, immunostaining analysis using antibodies against myosin heavy chain isoforms, and quantitation of CD31⁺ capillaries/fiber by immunostaining analysis, respectively. These results suggest that Zip14 mediates cancer-induced cachexia.

To rule out secondary effects of germline Zip14 loss, Zip14 levels in muscles were depleted by short-hairpin (sh), RNA-mediated knockdown and determined its effect on cancer-induced cachexia. Gastrocnemius muscles were transduced with an adeno-associated virus (AAV) expressing mCherry (to confirm successful transduction) in combination with either a shRNA targeting Zip14 (shZip14), or a scrambled control (shCon). A group of these mice were injected with C26m2 cancer cells and monitored metastasis and cachexia development, while remaining mice were used as non-tumor-bearing controls. Zip14 knockdown in muscles was confirmed by both qRT-PCR and immunostaining analysis. Consistent with the Zip14 knockout findings, Zip14 knockdown in muscles was also associated with a significant rescue of cancer-induced muscle atrophy. No differences in tumor burden, distribution of oxidative and glycolytic fibers, fiber-type, and vascularization were observed between the shCon and sh-Zip14 groups. In contrast, shZip14-expressing muscles from non-tumor-bearing mice did not exhibit a similar reduction in muscle atrophy as the tumor-bearing mice. These findings support that muscle-specific Zip14 expression is required for muscle wasting in the context of metastatic cancer.

Based on the induction of genes encoding zinc-binding proteins in cachectic muscles and the ability of ZIP14 to transport zinc in other tissues, ZIP14 imports zinc into muscle cells. Mice harboring C26m2 and 4T1 metastases showed aberrant accumulation of zinc in cachectic muscles (gastrocnemius, tibialis anterior, diaphragm, quadriceps, soleus, EDL, and heart) by both inductively-coupled-plasma mass spectrometry, and intracellularly within isolated single muscle fibers by laser-ablation inductively-coupled-plasma mass spectrometry (LA-ICP-MS). In contrast, tumor-bearing Zip14-null mice showed no additional zinc accumulation in muscles compared to non-tumor-bearing Zip14-null mice. To determine whether overexpression of Zip14 can augment zinc uptake in muscle cells, either GFP (control) or Zip14 was expressed in C2C12 myoblasts.

Zinc was added to the culture media and measured its uptake using a FluoZin-3 fluorescence based assay. Irrespective of differentiation status, Zip14-expressing C2C12 cells showed a marked increase in zinc uptake, as measured by its reduction in culture media. These results demonstrate that Zip14 likely functions as a zinc transporter in muscle cells.

If Zip14-mediated zinc uptake promotes the development of cancer-induced cachexia, then excess zinc should exacerbate muscle wasting in the context of cancer. In the absence of tumors, zinc supplementation had no detrimental effect on the growth kinetics of Zip14-wild-type and knockout mice. Strikingly, excess zinc induced a substantial acceleration in body weight loss and an increase in muscle atrophy in Zip14-wild-type, but not Zip14-knockout, tumor-bearing mice. No changes in food or water intake, behavior, liver or kidney function were observed in tumor-bearing mice with excess zinc supplementation thereby ruling out a role for acute toxicity effects. The Zip14/zinc-mediated cachexia was also not secondary to altered tumor burden since tumor volume was comparable between Zip14 WT and KO mice. These results indicate that excess zinc promotes muscle wasting in mice specifically in the presence of Zip14 and cachexia-inducing metastatic tumors.

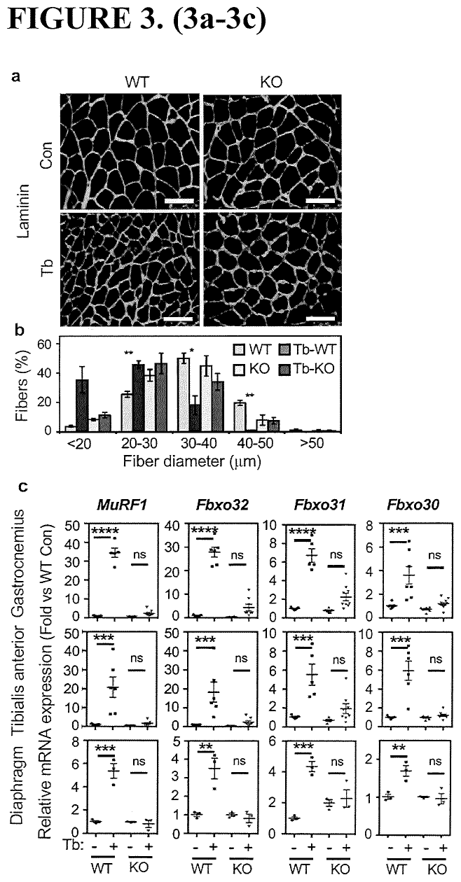

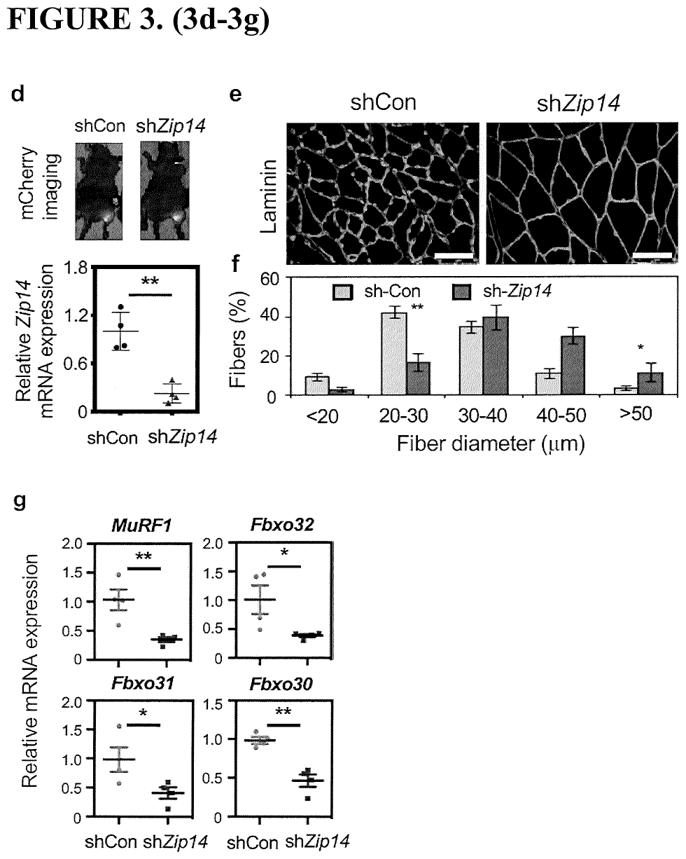

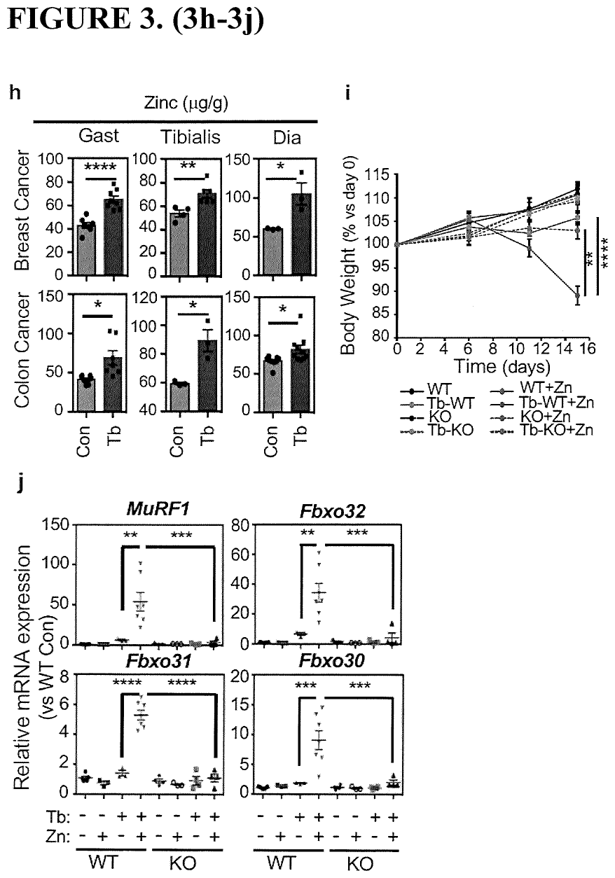

FIGS. 3a and 3b: Representative immunofluorescence images (a) and associated morphometric analysis (b) of cross-sections of gastrocnemius muscles harvested from mice at 5 weeks post 4T1-cell injection (n=3-4 mice per group). Scale bars, 50 pm. (a) Sections immunostained with antibody against laminin (shown in green) and stained with DAPI (shown in blue) (b) Morphometric analysis is depicted as the distribution frequency of fiber size categorized by fiber diameter.

FIG. 3c: qRT-PCR analysis of the indicated atrophy markers in gastrocnemius, tibialis anterior or diaphragm muscles from Zip14 wild-type (WT) and Zip14-knockout (KO) mice, with or without 4T1 tumor-cell injection, collected five weeks post injection. n=5-9 per group for gastrocnemius muscles, n=5-7 per group for tibialis anterior muscles, and n=3 for diaphragm muscles. Data were normalized to non-tumor-bearing Zip14 WT mice.

FIG. 3d: AAV vectors expressing mCherry as well as a shRNA targeting either a scrambled sequence (shCon) or Zip14 (shZip14) were injected intramuscularly into the gastrocnemius muscles and monitored by fluorescence imaging. A representative image taken five weeks after injection of AAV particles intramuscularly is shown in the upper panel. C26m2 cancer cells were then subcutaneously injected, and metastasis was monitored as previously described. Muscles were collected five weeks after tumor-cell injection, and Zip14 expression was determined by qRT-PCR analysis (lower panel). n=4 per group. Data were normalized to shCon.

FIGS. 3e and 3f: Representative immunofluorescence staining images of laminin (e) and morphometric analysis of muscle size in gastrocnemius muscles from C26m2 tumor-bearing (Tb) mice injected with either shCon or shZip14 (f). (e) Sections immunostained with antibody against laminin (shown in green) and stained with DAPI (shown in blue). Scale bars, 50 pm. (f) Morphometric analysis is depicted as the distribution frequency of fiber size categorized by fiber diameter. n=3-4 per group.

FIG. 3g: qRT-PCR analysis of the indicated genes in gastrocnemius muscles from C26m2 tumor-bearing (Tb) mice shown in (d), n=4 per group. Data were normalized to shCon.

FIG. 3h: Zinc levels in gastrocnemius, tibialis anterior and diaphragm muscles (μg/g of dry weight) determined by inductively-coupled-plasma-mass-spectrometry (ICP/MS) analysis from either non-tumor-bearing control mice or mice bearing either 4T1 or C26m2 metastases collected at 5 weeks post tumor-cell injection. n=3-8 per group for 4T1 model, and n=3-10 per group for C26m2 model.

FIGS. 3i and 3j: Body weight analysis (i) and qRT-PCR analysis of the indicated genes in TA muscles (j) of Zip14 wild-type (WT) and Zip14 knockout (KO) mice, either injected with C26m2 cancer cells or left uninjected as non-tumor-bearing controls. Mice were subdivided into two groups on the day of tumor cell injection and treated with either normal or zinc-supplemented drinking water for the indicated number of days in (i). TA muscles were harvested for qRT-PCR analysis after 15 days on zinc-supplemented water in (j). n=7-8 for WT, n=4-5 for KO (i) and n=3-7 for WT, n=3-4 for KO (j). Data were normalized to non-tumor-bearing Zip14 WT mice on normal water.

Error bars represent SEM. p values determined by two-tailed, unpaired Student's t-test in (d, g and h), one-way ANOVA with post-hoc Tukey's test in (c, i and j), and Welch's t-test in (f). ns, not significant. *p<0.05, **p<0.01, ***p<0.001 and ****p<0.0001. Con, control; Tb, tumor-bearing.

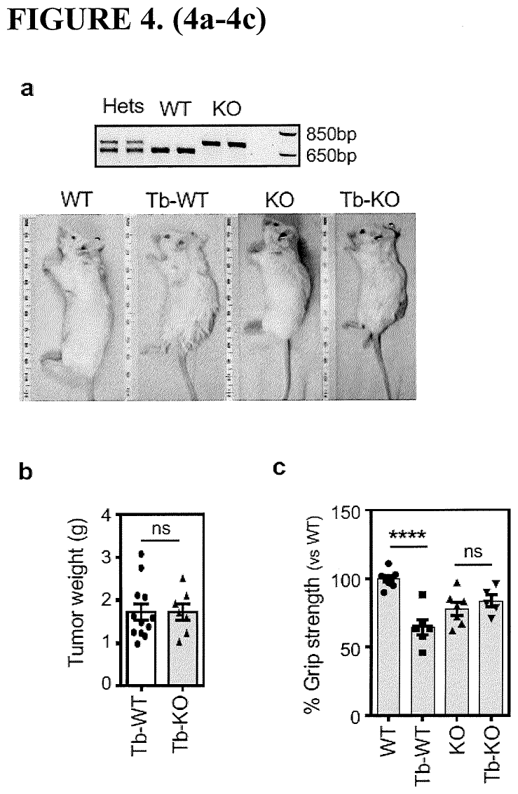

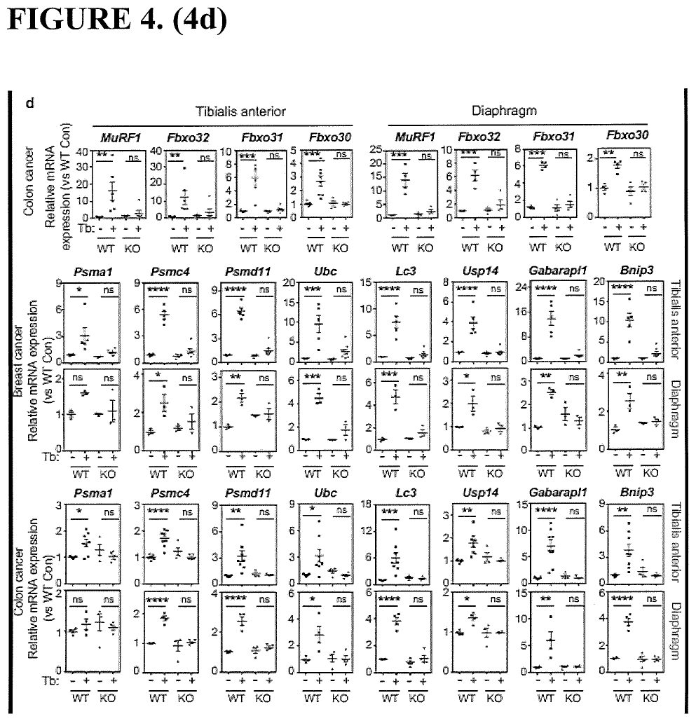

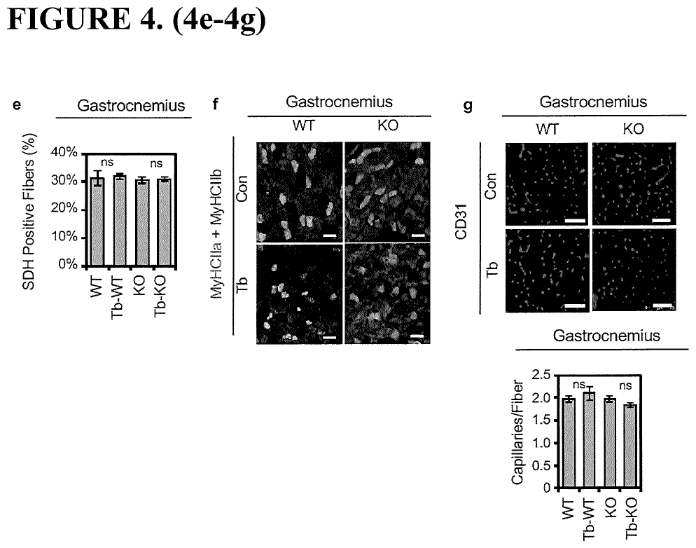

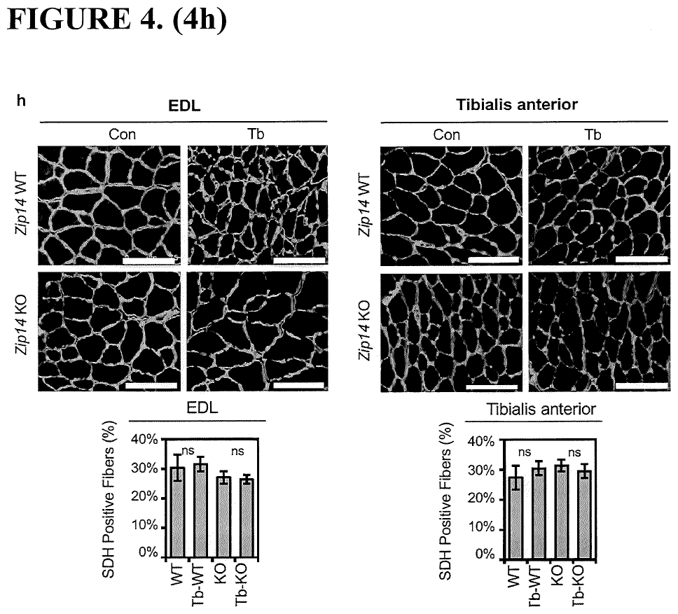

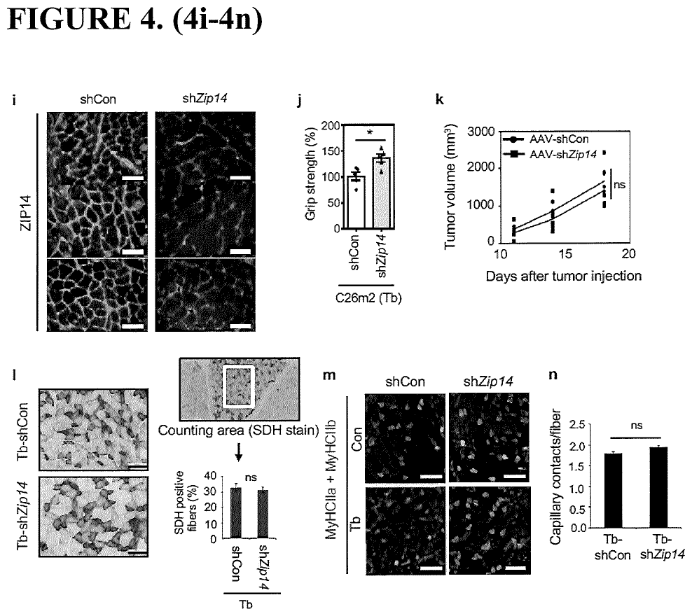

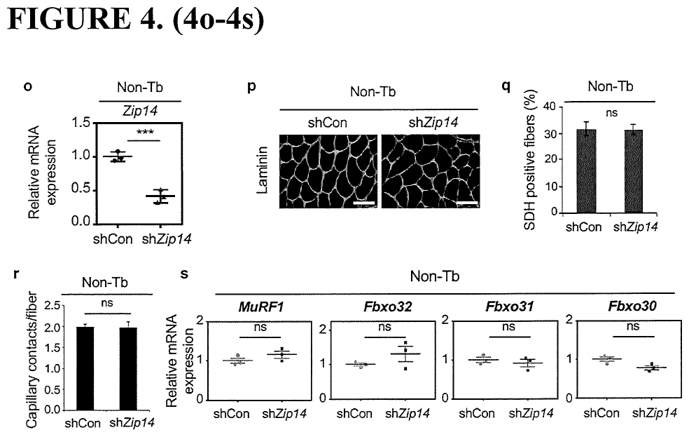

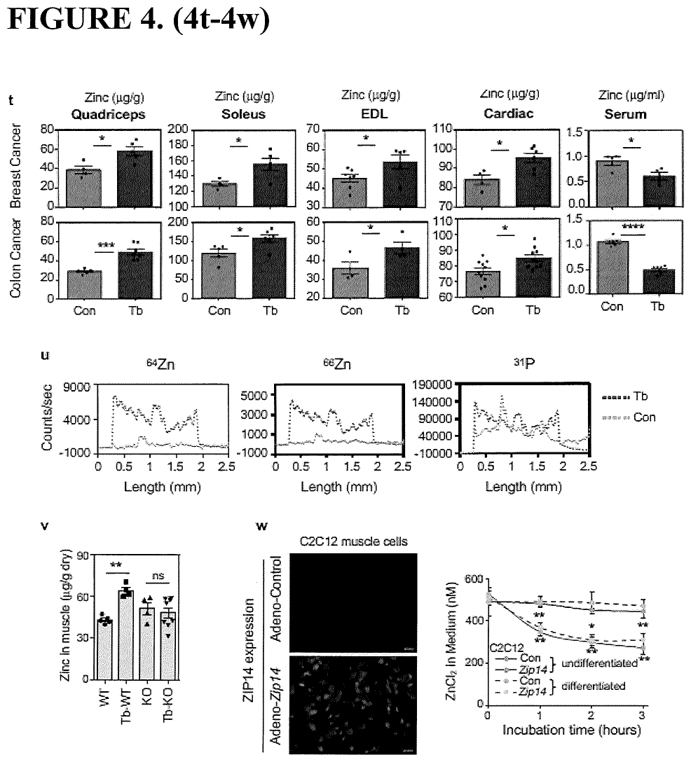

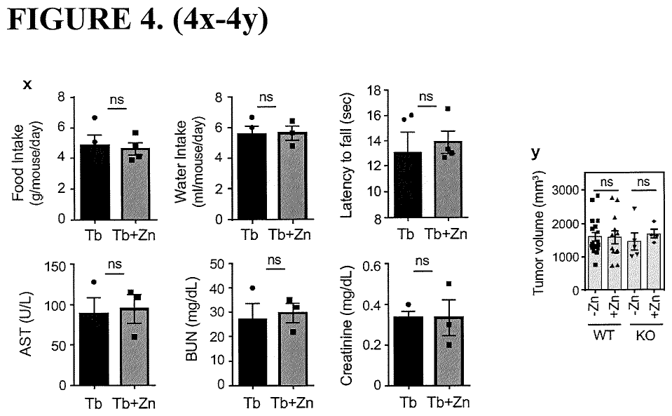

FIG. 4 shows the analysis results of Zip14-WT and Zip14-KO to demonstrate that Zip14 loss reduces metastatic-cancer-induced muscle atrophy. FIG. 4a: Genotyping analysis for validation of germline Zip14 KO mice. Zip14 heterozygous (Hets, lanes 1 and 2), Zip14-WT (lanes 3 and 4) and Zip14-KO mice (lanes 5 and 6). Representative images of non-tumor-bearing and tumor-bearing Zip14-WT or Zip14-KO mice are shown below. FIG. 4b: Tumor weight of Zip14-WT and Zip14-KO mice with 4T1 tumor burden (Tb). n=12 for Zip14-WT mice, and n=7 for Zip14-KO mice with 4T1 tumor burden. FIG. 4c: Normalized hind limb grip strength of Zip14-WT and Zip14-KO mice with 4T1 tumor burden (Tb) or age-matched non-tumor-bearing controls. n=5-8 per group. FIG. 4d: (Top panels) qRT-PCR analysis of atrophy-associated markers MuRF1, MAFbx/Fbxo32, Fbxo31 and Musa1/Fbxo30 in tibialis anterior and diaphragm muscles from C26m2 tumor-bearing Zip14-WT or Zip14-KO mice and non-tumor-bearing control mice. n=4-8 per group for tibialis anterior muscles, and n=4 per group for diaphragm muscles. (Middle and lower panels) qRT-PCR analysis of genes associated with ubiquitination (Ubc and Usp14), UPS (Psmal, Psmc4 and Psmd11) and autophagy (Lc3, Gabarapll and Bnip3) pathways in tibialis anterior and diaphragm muscles from Zip14-WT and Zip14-KO mice with 4T1 (Tb, middle panels) or C26m2 (Tb, lower panels) metastases, compared with the age-matched, non-tumor-bearing controls. n=4-8 per group for tibialis anterior and n=3 per group for diaphragm muscles from the 4T1 model, and n=4-8 per group for tibialis anterior and n=4 per group for diaphragm muscles from the C26m2 model. Data were normalized to non-tumor-bearing Zip14-WT control mice. FIG. 4e: Quantitation of the percentage of succinate dehydrogenase (SDH) positive fibers in gastrocnemius muscles from 4T1-tumor-bearing Z/′p74-WT or Zip14-KO mice and age-matched control mice. n=3 per group. FIG. 4f: Representative immunofluorescence images of MyHC IIa (green) and MyHC IIb (red) expression in gastrocnemius muscles from Zip14-WT and Zip14-KO mice with or without 4T1-tumor-cell injection. Scale bars, 50 pm. FIG. 4g: (Top panel) Representative immunofluorescence images of CD31 expression in gastrocnemius muscles from 4T1-tumor-bearing Zip14-WT or Zip14-KO mice and age-matched non-tumor-bearing control mice. (Bottom panel) Quantitation of CD31-positive capillaries per fiber. n=3 per group. Scale bars, 50 pm. FIG. 4h: Representative immunofluorescence images of laminin (top panels) and quantitation of the percentage of SDH-positive fibers (bottom panels) in EDL and tibialis anterior muscles from 4T1-tumor-bearing Zip14-WT or Zip14-KO mice and age-matched, non-tumor-bearing control mice. Scale bars, 50 pm. For SDH quantitation, n=3 per group. FIG. 4i: Representative immunofluorescence images of Zip14 in gastrocnemius muscles isolated from C26m2-tumor-bearing mice injected intramuscularly with either shCon or shZip14. n=3 per group. Green, Zip14; blue, DAPI. Scale bars, 50 pm.

FIGS. 4j and 4k: Normalized hind limb grip strength measurement (j) and tumor volume (k) in mice injected with either shCon or shZip14 in their gastrocnemius muscles followed by C26m2-tumor-cell injection. n=4-5 mice per group. Tb, tumor-bearing. FIG. 4l: Representative images of SDH immunostaining in gastrocnemius muscles from C26m2-tumor-bearing mice injected intramuscularly with either shCon or shZip14, and the quantitation of the percentage of SDH-positive fibers shown on the right. Scale bars, 100 pm. n=3 per group. FIG. 4m: Representative immunofluorescence images of MyHC IIa (green) and MyHC IIb (red) in gastrocnemius muscles from mice injected intramuscularly with either shCon or shZip14 from C26m2-tumor-bearing or age-matched control mice. Scale bars, 100 pm. FIG. 4n: Quantitation of the number of capillaries per muscle fiber in gastrocnemius muscles from C26m2-tumor-bearing mice injected intramuscularly with either shCon or shZip14. n=3 per group. FIG. 4o: qRT-PCR analysis of Zip14 in gastrocnemius muscles from non-tumor-bearing mice injected with either shCon or shZip14 in their gastrocnemius muscles. n=3 per group. Data were normalized to shCon. FIG. 4p: Representative immunofluorescence images of laminin in gastrocnemius muscles from non-tumor-bearing mice injected intramuscularly with either shCon or shZip14. Scale bars, 50 pm. FIGS. 4q and 4r: Quantitation of the percentage of SDH-positive fibers (q) and the number of capillaries per fiber (r) in gastrocnemius muscles from non-tumor-bearing mice injected intramuscularly with either shCon or shZip14. n=3 per group. FIG. 4s: qRT-PCR analysis of MuRF1, MAFbx/Fbxo32, Fbxo31 and Musa1/Fbxo30 in gastrocnemius muscles from non-tumor-bearing mice injected intramuscularly with either shCon or shZip14. n=3 per group. Data were normalized to shCon. FIG. 4t: Zinc metal-ion levels determined by ICP/MS analysis in quadriceps, soleus, EDL, cardiac muscles (μg/g of dry weight) and serum (μg/ml of serum) from mice with 4T1 tumor metastases (upper panel) or C26m2 tumor metastases (lower panel) and respective age-matched, non-tumor-bearing controls. n=4-7 per group for the 4T1 model and n=4-10 per group for the C26m2 model.

FIG. 4u: Intracellular Zn and P levels within single muscle fibers isolated from non-tumor-bearing mice or mice bearing C26m2 metastases by LA-ICP Mass spectrometry are shown. Single EDL muscle fibers were isolated, mounted on slides and air-dried. Slides were placed in sealed ablation cell and ablated along a single fiber for about 2 mm with a speed of 5 pm/sec. Individual muscle fiber data was subtracted from a blank line on the same slide with same dimension, size and parameters as the sample line. Data representative of three independent experiments with n=3 mice per group, 3-5 fibers per mouse. FIG. 4v: Zinc metal-ion levels (μg/g of dry weight) determined by ICP/MS analysis in gastrocnemius muscle of 4T1-tumor-bearing Zip14-WT or Zip14-KO mice compared to non-tumor bearing littermates. n=4-8 mice per group. FIG. 4w: (Left) Representative immunofluorescence images of Zip14 (red) in C2C12 muscle cells infected with adenovirus expressing either GFP (Adeno-Control) or Zip14 (Adeno-Zip14). Blue, DAPI. Scale bars, 50 pm. (Right) Adeno-Control (Con) and Adeno-Zip14 (Zip14) infected C2C12 muscle cells were treated with 0.5 μM ZnCI₂, and the reduction of zinc in the medium over time was measured by the FluoZin-3 fluorescence-based assay. Undifferentiated C2C12 or 3-day differentiated C2C12 cells were used for this assay. Data is representative of three independent experiment FIG. 4x: Food and water intake and behavioral coordination tests by rotarod (upper panel), liver and kidney function tests (AST, BUN, and creatinine, lower panel) in C26m2-tumor-bearing mice with and without zinc supplemented water. n=3-4 per group. FIG. 4y: Tumor volume of Zip14-WT or -KO mice injected with C26m2 cancer cells and treated with either regular water (−Zn) or zinc-supplemented water (+Zn). n=18 for Tb-WT−Zn; n=12 for Tb-WT+Zn; n=5 for Tb-KO−Zn; n=4 for Tb-KO+Zn.

Error bars represent SEM. p values were determined by two-tailed, unpaired Student's t-test in (b, j, k, I, n, o and q-t, x) and by one-way ANOVA with post-hoc Tukey's test in (c-e, g, h, v, w and y). ns, not significant. *p<0.05, **p<0.01, ***p<0.001 and ****p<0.0001; Con, non-tumor bearing control; Tb, tumor-bearing.

### Example 5. Muscle Homeostasis and Atrophy

To understand how excess zinc might perturb muscle homeostasis and mediate muscle atrophy, different cell types in cachectic muscles were examined to observe the expression of Zip14. Progenitor subpopulations were purified in muscles by magnetic and flow-cytometry-assisted sorting and muscle sections were immunostained with an antibody against Zip14. In muscles from both C26m2 and 4T1 metastasis models, Zip14 was specifically induced in CD45⁻/CD31⁻/Sca1⁻/CD34⁺/α7-integrin⁺ cells which is the muscle satellite-cell population associated with cachexia, and confirmed this finding in human muscle satellite cells expressing PAX7. Zip14 expression was also observed in mature, differentiated myofibers from cachectic muscles in the C26m2 and 4T1 metastasis models. Therefore, Zip14-mediated zinc accumulation negatively impacted both the process of muscle-cell differentiation and the function of differentiated muscle fibers.

Normal muscles respond to muscle injury by activating and proliferating muscle progenitor cells into myoblasts that differentiate to regenerate new muscle fibers. In contrast, in conditions associated with muscle atrophy including in cancer, muscles are damaged followed by proliferation of muscle progenitor cells, which eventually fail to differentiate. Experiments were performed to test whether aberrant Zip14 upregulation and consequent zinc influx in muscle progenitor cells could block normal differentiation using C2C12 myoblasts and primary myoblasts. Both cell types were infected with adenovirus expressing either GFP (Adeno-Con) or Zip14 (Adeno-Zip14). Each group differentiated normally in the absence of zinc as assessed by expression of myosin heavy chain (MyHC) and cellular morphology. In contrast, in the presence of zinc, the differentiation of Zip14-expressing myoblasts was selectively blocked with no loss in viability. These findings suggest that Zip14-mediated zinc uptake in muscle progenitor cells interferes with muscle-cell differentiation.

Myoblasts deficient in the myogenic transcription factors MyoD and Mef2 can proliferate, but are unable to differentiate. It is possible that excess zinc could repress the levels, or activity of, myogenic transcription factors to block muscle-cell differentiation. Treatment of Zip14-expressing C2C12 myoblasts with zinc led to transcriptional repression of MyoD, Mef2c, and Myf5 but not Cyclin D1, which controls proliferation and cell-cycle exit of myoblasts. Furthermore, GSEA using HALLMARK-MYOGENESIS gene sets querying the cachexia signature derived from C26m2 and 4T1 metastasis models was supportive of repressed myogenesis in cachectic muscles. Consistently, MyoD and Mef2c expression was downregulated in cachectic muscles from C26m2 and 4T1 metastasis models, compared to non-tumor bearing controls. These findings identified a potential link between Zip14-induced zinc accumulation in muscle progenitor cells and impaired muscle regeneration in the context of metastatic cancer.

Myofibrils constitute the organizational units in muscle with aligned thick and thin filaments that facilitate muscle contraction. Myofibrillar proteins comprise over 70% of muscle proteins, and their reduced synthesis or loss negatively affects fiber size and function. In order to test whether Zip14-mediated zinc influx affects myofibrillar protein levels, Zip14-expressing and control myoblasts were differentiated into myotubes. Myotubes were treated with zinc for 24 hours and myofibrillar proteins were extracted using high-salt lysis method. A striking loss in MyHC protein was observed in Zip14-expressing myotubes treated with zinc. In contrast, the thin filament proteins skeletal actin, tropomyosin and troponin, the intermediate filament protein desmin, and the thick filament protein myosin light chain (MyLC) remained unchanged under these conditions. Furthermore, fractionation of muscle proteins showed that both the soluble and myofibrillar fractions of MyHC predominantly decreased in Zip14-expressing myotubes with zinc treatment over other myofibrillar proteins. These results suggest that ZIP14-mediated zinc accumulation induces the loss of both soluble and sarcomeric MyHC in mature muscle cells.

The ubiquitin-proteasome system (UPS) is one of the central pathways that regulate MyHC turnover in muscle atrophy states, and loss of MyHC is associated with loss of muscle mass and function during cancer cachexia. Therefore, it was tested whether the UPS promotes MyHC loss in the context of ZIP14-mediated zinc influx and cancer-induced muscle wasting. MyHC loss in differentiated C2C12 cells was associated with upregulation of MuRF1, Psmal, Psmc4, Psmd11 and Ubc UPS pathway genes and could be blocked by the proteasome inhibitor, MG132. Consistent with in-vitro studies, MyHC levels in cachectic muscles from the metastasis models were restored to normal in response to either Zip14 knockdown (C26m2 model) or loss (4T1 model) with no changes in expression of the other myofibrillar proteins examined. To confirm the specificity of MyHC regulation in vivo by Zip14, Zip14 in Zip14-deficient muscles were re-expressed. The gastrocnemius muscles of Zip14 germline knockout mice with AAV-expressing Zip14 or mCherry were transduced as a control. 4T1 cancer cells were subcutaneously implanted to evaluate the effects of Zip14 re-expression in muscle during cancer-induced cachexia. Re-expression of Zip14 in muscles reestablished the muscle atrophy phenotype in tumor-bearing Zip14-deficient mice resulting in significant MyHC loss with no changes in fiber type or vascularization. These results suggest that Zip14 mediates muscle atrophy through MyHC loss in cancer.

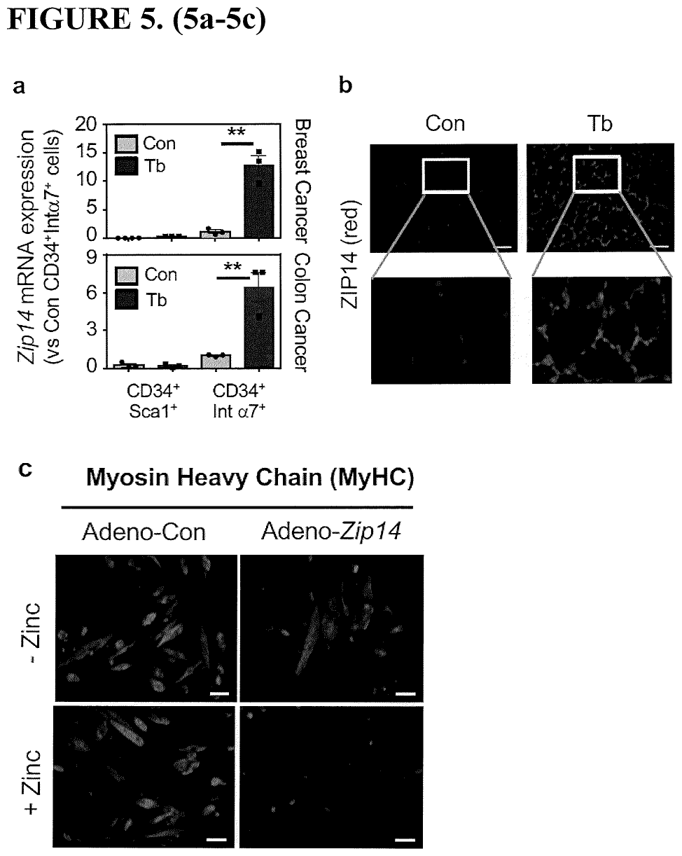

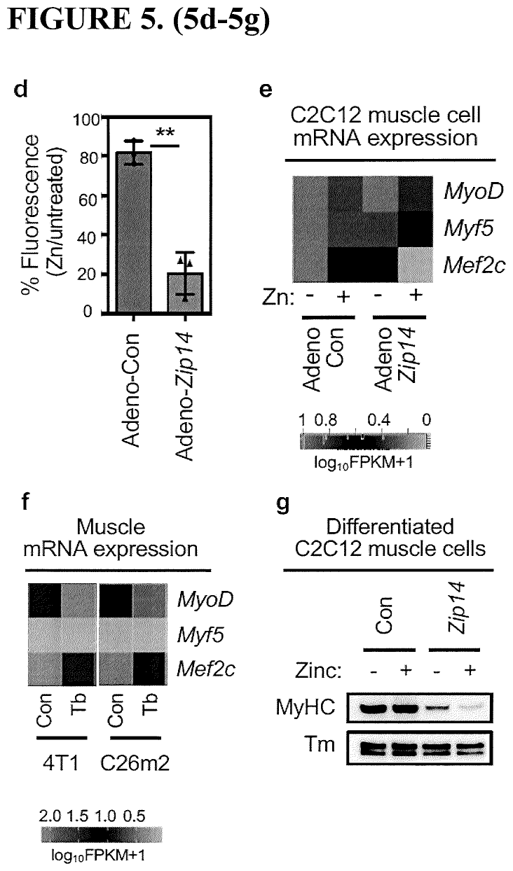

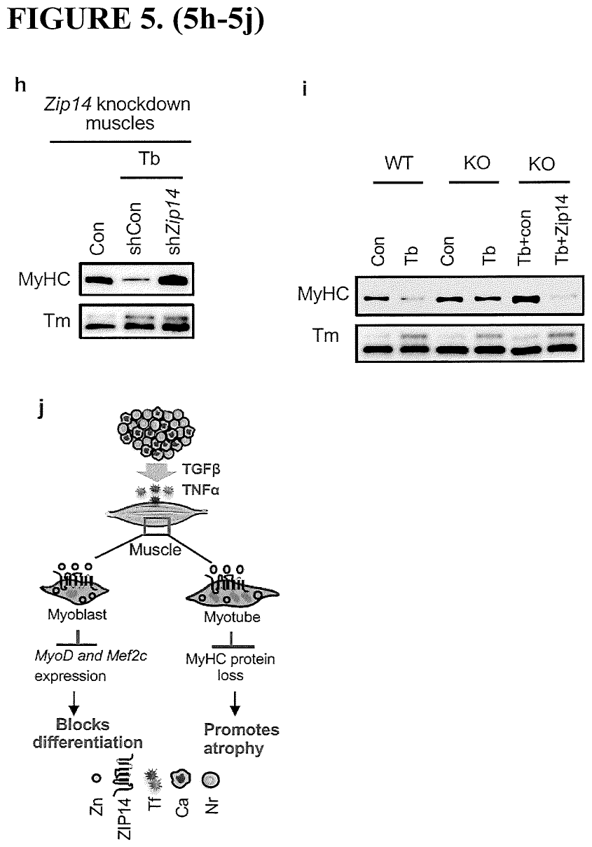

FIG. 5 shows the analysis results of non-tumor-bearing control mice (Con) or mice bearing 4T1 or C26m2 metastases to demonstrate that Zip14-mediated zinc accumulation blocks muscle-cell differentiation and induces myosin heavy chain loss. FIG. 5a: qRT-PCR analysis of Zip14 expression in purified muscle progenitor subpopulations from either non-tumor-bearing control mice (Con) or mice bearing 4T1 or C26m2 metastases (Tb) harvested five weeks after tumor-cell injection. CD45⁻CD31⁻CD34⁺Sca1⁺ and CD45⁻CD31⁻ CD34⁺ integrin-α7⁺ cells were purified from gastrocnemius muscles using a combination of magnetic and flow-cytometry-assisted sorting. FIG. 5b: Zip14 immunofluorescence analysis using muscle sections from either non-tumor-bearing control mice or mice bearing C26m2 metastases five weeks after tumor-cell injection. Zip14 (red), DAPI (blue). FIGS. 5c and 5d: Immunofluorescence analysis showing myosin heavy chain (MyHC) expression in C2C12 myoblasts infected with adenovirus expressing either control (Adeno-Con) or Zip14 cDNA (Adeno-Zip14) and differentiated for 6 days, either with 0 or 50 μM ZnCI₂ (zinc) replenished daily. FIG. 5 e: qRT-PCR analysis of MyoD, Myf5 and Mef2c in untreated and zinc-treated C2C12 cells expressing either Adeno-Con or Adeno-Zip14, represented as a heatmap. Adenovirus-infected C2C12 cells were differentiated for 2 days and then treated with either 0 or 50 μM ZnCI₂ (zinc) for 24 hours. FIG. 5f: RNA-Seq analysis of MyoD, Myf5 and Mef2c shown as heatmap comparing TA muscles from non-tumor-bearing control mice to mice bearing 4T1 or C26m2 metastases, collected 5 weeks post tumor-cell injection. FIG. 5g: MyHC and tropomyosin (Tm) protein expression by immunoblot analysis in C2C12 cells infected with adenovirus expressing either control or Zip14 cDNA and differentiated for 3 days followed by treatment with either 0 or 50 μM ZnCI₂ for 24 hours. FIG. 5h: Immunoblot analysis probing for MyHC and Tm in gastrocnemius muscles from mice intramuscularly injected with adeno-associated virus expressing either shCon or shZip14 and subsequently injected with C26m2 cancer cells. Age-matched, non-tumor-bearing mice were used as a control. FIG. 5i: Immunoblot analysis probing for MyHC and Tm in gastrocnemius muscles from the indicated groups. Muscles were isolated from Zip14-WT and Zip14-KO mice with (Tb) or without (Con) 4T1 metastases. Another cohort of Zip14-KO mice were injected intramuscularly with AAV-Con (mCherry) or AAV-Zip14 in the gastrocnemius muscle and injected four weeks later with 4T1 tumor cells. FIG. 5j: Working model: During cancer progression and metastasis, cytokines such as TNFα and TGFβ upregulate the expression of Zip14, a metal ion transporter, in muscle cells. This causes an aberrant accumulation of zinc in muscle. Zip14 expression and zinc uptake in muscle progenitor cells then represses key myogenic genes such as MyoD and Mef2c, blocks muscle differentiation, and reduces myosin heavy chain expression. Zip14-mediated zinc overload in skeletal muscle promotes metastatic-cancer-induced muscle atrophy through loss of myofibrillar protein. Tf, tumor factors; Ca, cancer cells; Nr, normal cells.

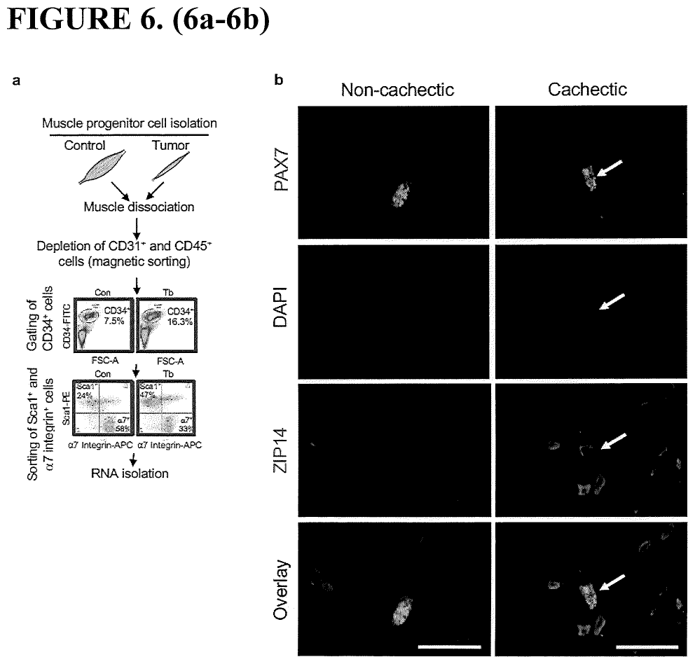

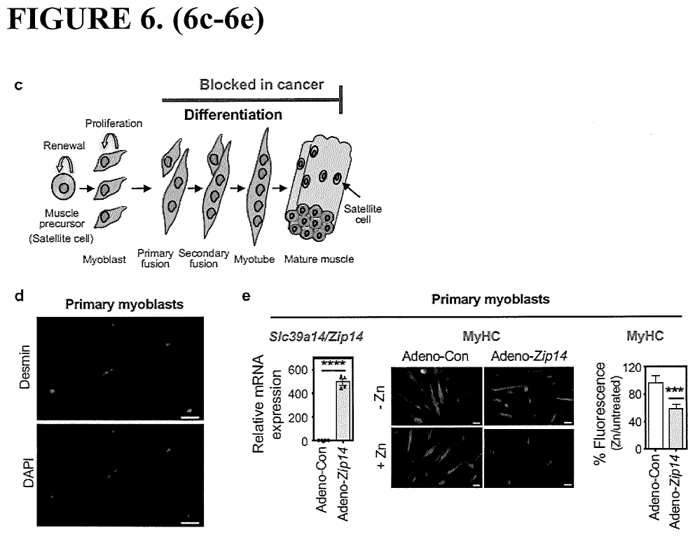

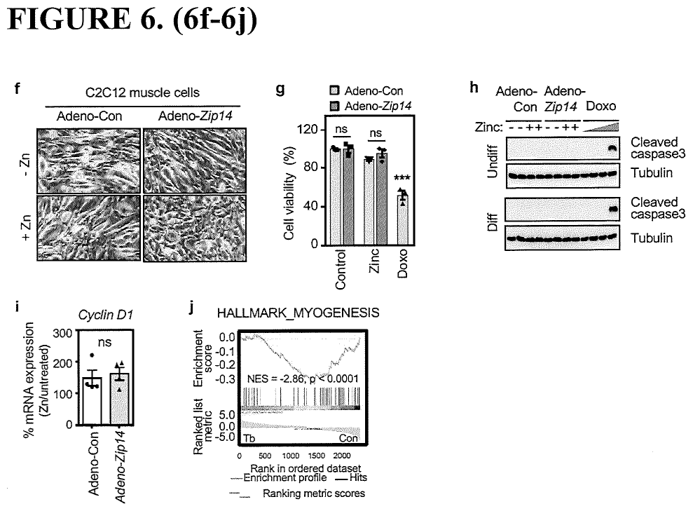

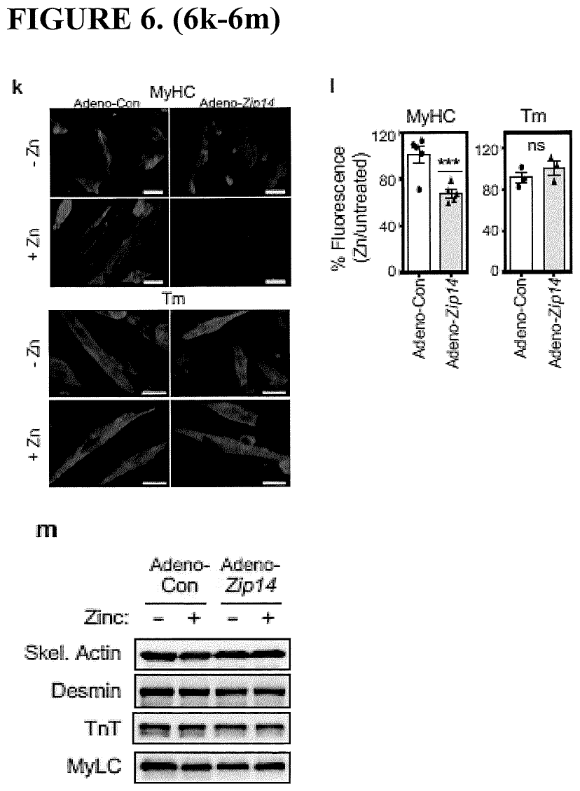

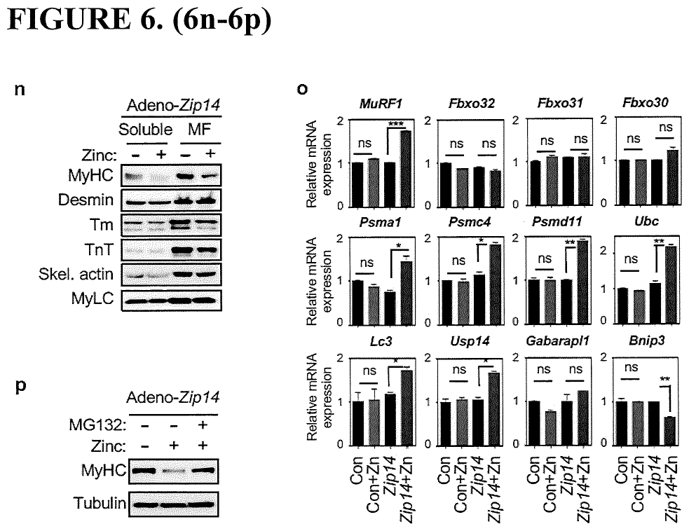

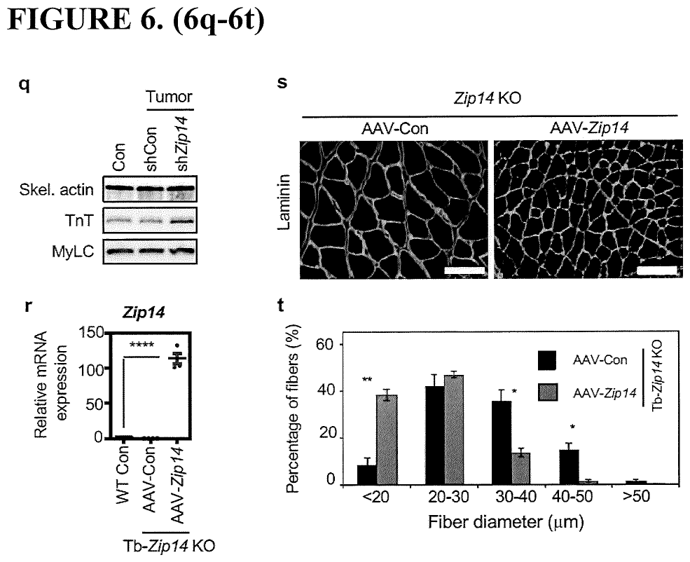

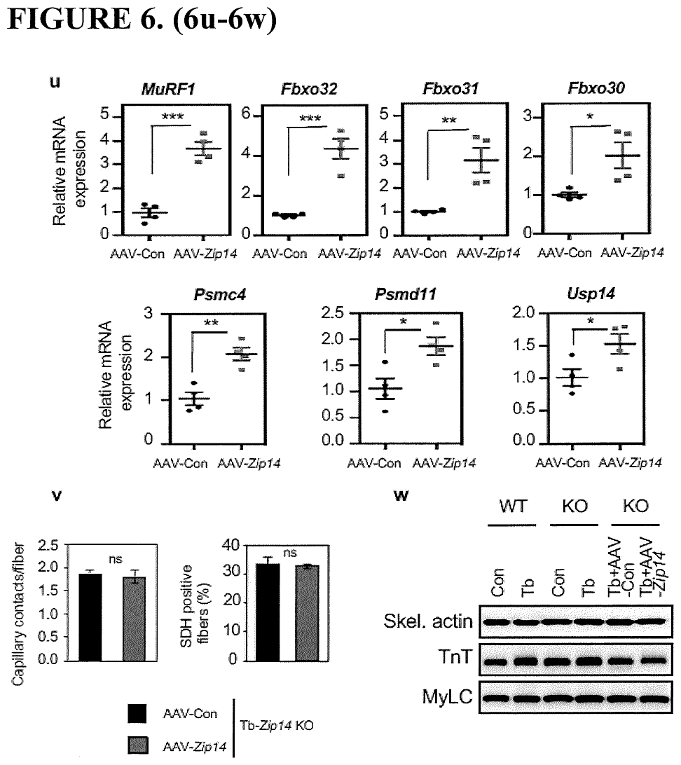

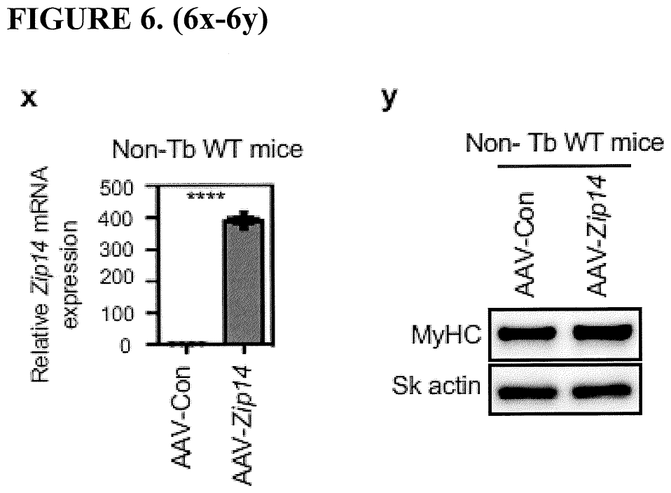

FIG. 6 shows the analysis results of myosin heavy chain loss induced by Zip14-mediated zinc uptake in muscle cells. FIG. 6a: Schematic representation of isolation and representative flow cytometry analysis of the indicated muscle progenitor populations in gastrocnemius muscles from mice bearing either 4T1 or C26m2 metastases (Tb) collected 5 weeks after tumor resection and compared with age-matched, non-tumor-bearing control mice (Con). FIG. 6b: Representative immunofluorescence images of psoas muscle sections using antibodies against PAX7 (shown in green) and Zip14 (shown in red) from non-cachectic (left) or cachectic (right) metastatic cancer patients. Blue, DAPI. White arrows indicate satellite cell expressing both PAX7 and Zip14. FIG. 6c: Schematic model based on previous studies showing the process of muscle differentiation. In response to injury of normal muscles, quiescent satellite cells are activated, proliferate and become committed as myoblasts. Once committed, myoblasts exit the cell cycle and fuse to form multinucleated myofibers. These new myofibers contribute to mature muscle mass. This process is thought to be blocked in cancer, where muscles fail to regenerate despite satellite-cell activation and proliferation. FIG. 6d: Representative immunofluorescence images using antibody against desmin (red) to identify primary myoblasts in culture. FIG. 6e: Differentiation of control and Zip14-expressing primary myoblasts with and without zinc treatment. (Left panel) Primary myoblasts were infected with adenovirus expressing either GFP control (Adeno-Con) or Zip14 (Adeno-Zip14), and relative Zip14 expression levels were measured by qRT-PCR analysis two days after infection. (Middle and right panels) Primary myoblasts were differentiated for 2 days followed by treatment with either 0 or 50 μM ZnCI₂ for 24 h. Differentiation was then quantified by MyHC immunofluorescence staining. FIG. 6f: Representative bright-field images of C2C12 cells infected with adenovirus expressing either GFP (Adeno-Con) or Zip14 (Adeno-Zip14) and differentiated for 6 days either in the presence of 0 or 50 μM ZnCI₂ replenished daily. FIGS. 6g and 6h: C2C12 cells expressing either GFP (Adeno-Con) or Zip14 (Adeno-Zip14) were differentiated for 3 days and treated with either 0 or 50 μM ZnCI₂ for 24 h. Cell viability was then measured by MTS assay (g) or by immunoblot analysis for cleaved caspase-3 with tubulin as a loading control (h). C2C12 cells treated with doxorubicin (Doxo) chemotherapy was used as a positive control in both assays. Doxo was used at a dose of 2.5 pM in (g) and 0 pM, 0.25 pM (duplicate), and 2.5 pM in (h). Undiff and Diff represent undifferentiated and differentiated C2C12 muscle cells, respectively. FIG. 6i: C2C12 cells expressing either GFP (Adeno-Con) or Zip14 (Adeno-Zip14) were differentiated for two days and then treated with 0 or 50 μM ZnCI₂ for 24 h. Relative CyclinDI expression was then determined by qRT-PCR analysis. FIG. 6j: GSEA using HALLMARK-MYOGENESIS gene set querying RNA-Seq gene signatures from cachectic muscles. Pre-ranked GSEA was performed on standalone GSEA (v2.2.2) using the hallmark genes set h.all.v5.2.symbols.gmt for the differentially expressed genes obtained from C26m2 (common to 4T1), ranked by their log 2 fold change (FC) values with significant p and q values.

FIGS. 6k and 6l: Representative immunofluorescence images (k) and quantitation (l) of MyHC and tropomyosin (Tm). C2C12 cells expressing either GFP (Adeno-Con) or Zip14 (Adeno-Zip14) were differentiated for 3 days and then treated with 0 or 50 μM ZnCI₂ for 24 h before analysis. FIG. 6m: Immunoblot analysis of skeletal actin, desmin, troponin T and MyLC in C2C12 cells expressing either GFP (Adeno-Con) or Zip14 (Adeno-Zip14). Cells were differentiated for 3 days and subsequently treated with either 0 or 50 μM ZnCI₂ for 24 h before analysis. FIG. 6n: Immunoblot analysis of the indicated proteins derived from lysates that were fractionated to generate soluble and myofibrillar (MF) fractions from C2C12 cells expressing either GFP (Adeno-Con) or Zip14 (Adeno-Zip14) that were differentiated for 3 days and subsequently treated with either 0 or 50 μM ZnCI₂ for 24 h before analysis. FIG. 6o: qRT-PCR analysis of genes associated with ubiquitination (MuRF1, Fbxo32, Fbxo31, Fbxo30, Ubc, Usp14), UPS (Psmal, Psmc4, Psmd11) and autophagy (Lc3, Gabarapll, Bnip3) pathways from C2C12 cells expressing either GFP (Adeno-Con) or Zip14 (Adeno-Zip14) that were differentiated for 3 days and subsequently treated with either 0 or 50 μM ZnCI₂ for 24 h before analysis. FIG. 6p: Immunoblot analysis of MyHC in C2C12 cells expressing Zip14 (Adeno-Zip14) that were differentiated for 3 days and subsequently treated with either 0 or 50 μM ZnCI₂ for 24 h before analysis. As indicated, cells were treated with either vehicle or MG132 (50 μm) 3 hours prior to harvest. Tubulin served as a loading control. FIG. 6q: Immunoblot analysis for skeletal actin, troponin T and MyLC in gastrocnemius muscles from mice intramuscularly injected with AAV delivering either shCon or shZip14 and subsequently injected with C26m2 cancer cells. Non-tumor-bearing mice were used as a control.

FIG. 6r-6u: Zip14-KO mice were intramuscularly injected in the gastrocnemius muscles with AAV delivering either mCherry control (AAV-Con) or Zip14 (AAV-Zip14) and subsequently injected with 4T1 cancer cells 4 weeks after AAV injection. Muscles were then harvested five weeks after tumor-cell injection, (r) qRT-PCR analysis was performed for the indicated genes and normalized to AAV-Con. FIG. 6s: Representative immunofluorescence images of laminin expression shown in green and DAPI in blue. FIG. 6t: Morphometric analysis is depicted as the distribution frequency of fiber size categorized by fiber diameter. FIG. 6u: qRT-PCR analysis was performed for the indicated genes and normalized to AAV-Con. FIG. 6v: Quantitation of the capillary contacts/fiber and percentage of SDH-positive fibers are shown in the gastrocnemius muscles from Zip14-KO mice that were intramuscularly injected with AAV delivering either mCherry control (AAV-Con) or Zip14 (AAV-Zip14). FIG. 6w: Immunoblot analysis of skeletal actin, troponin T and MyLC in gastrocnemius muscles from the indicated groups. Muscles were isolated from Zip14-WT and Zip14-KO mice with (Tb) or without (Con) 4T1 metastases. A separate group of Zip14-KO mice were injected intramuscularly with AAV-Con (mCherry) or AAV-Zip14 and subsequently injected with 4T1 tumor cells. FIGS. 6x and 6 y: qRT-PCR analysis of Zip14 (x) and immunoblot analysis of MyHC and skeletal actin (y) from non-tumor-bearing mice that were intramuscularly injected in the gastrocnemius muscles with either AAV-mCherry (AAV-Con) or AAV-Zip14.

### Example 6: Development of Cachexia in the Lewis Lung Carcinoma Mouse Model

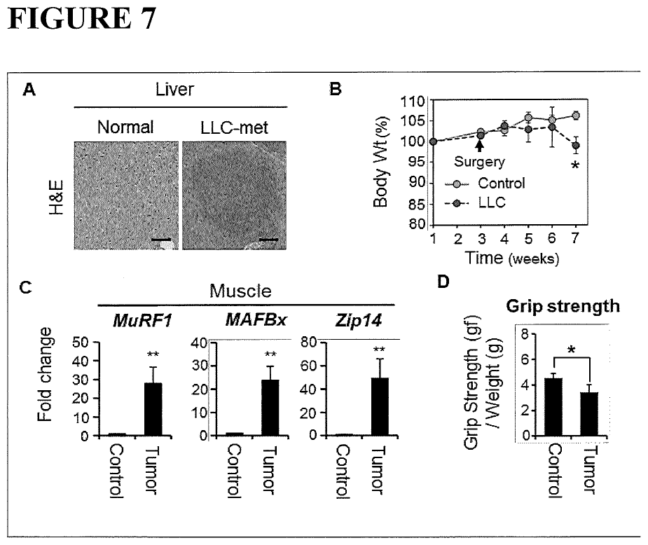

Progressive development of cachexia in the Lewis Lung carcinoma (LLC) mouse model of lung cancer metastasis was conducted. FIG. 7 shows the analysis results of the Lewis Lung carcinoma (LLC) mouse model of lung cancer metastasis. The results indicate the induction of Zip14 with cachexia. Histological analysis of metastasis in LLC model compared to normal liver (A), body weight change after LLC cells inoculation (B), relative MuRF1/MAFBx/Zip14 mRNA levels by qRT-PCR analysis in tibialis anterior (TA) limb muscle (C). Grip strength measurement (D). C and D comparing control (non-tumor bearing mice) and LLC tumor-bearing mice (tumor). Scale bars in A. n=8-15 mice per group; *p≤0.05, **p≤0.01

### Example 7: Development of Cachexia in the Pan02 Mouse Model of Pancreatic Cancer Metastasis

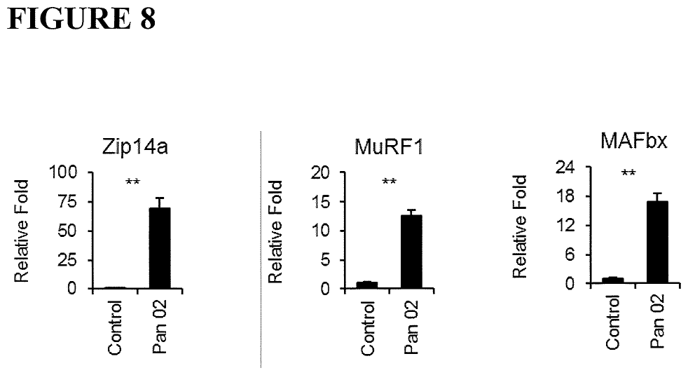

Progressive development of cachexia in the Pan02 mouse model of pancreatic cancer metastasis was conducted. FIG. 8 shows the analysis results of the Pan02 mouse model of pancreatic cancer metastasis indicating the induction of Zip14 with cachexia. FIG. 8 shows relative MuRF1/MAFBx/Zip14 mRNA levels by qRT-PCR analysis in tibialis anterior (TA) limb muscle comparing control (non-tumor bearing mice) and Pan02 tumor-bearing mice injected with 100,000 murine pancreatic cancer cells (Pan02 from NCI) into arterial circulation. Muscles were collected upon cachexia development. Scale bars in A. n=3 mice per group; **p≤0.01

### Example 8: Zinc Chelation Reduces Muscle Wasting in Tumor-Bearing Mice

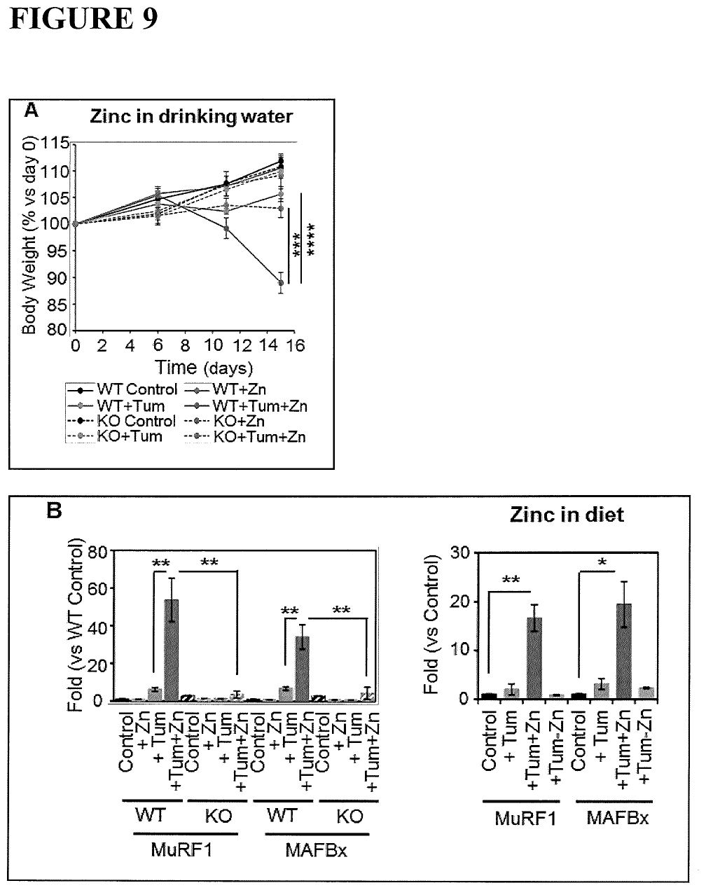

The effects of zinc chelator injection were analyzed using healthy or C26m2 tumor bearing mice. Excess zinc supplementation exacerbates muscle wasting, while zinc chelation reduces muscle wasting in tumor-bearing mice. FIG. 9 shows the analysis results of zinc chelation treatments for healthy or C26m2 tumor bearing mice treated with regular or zinc-enriched water with zinc chelator injection. (A-B) Healthy or C26m2 tumor bearing mice of the indicated genotypes (Zip14 WT or KO) were either treated with regular or zinc-enriched water (25 mM) from the day of tumor cell injection and muscles were collected after 16 days. Body weight analysis (A) and relative MuRF1/MAFBx mRNA expression levels indicative of muscle wasting (B) are shown. C. Mice were injected with 4T1 tumor cells and were divided into 3 groups, one was provided with regular diet with 86 ppm zinc, second group with 1000 ppm zinc-supplemented diet (high zinc diet) and third group with regular diet with 86 ppm zinc but with zinc chelator injected daily with Sodium diethyldithiocarbamate trihydrate (DEDTC) at 150 mg/kg body weight injected 7 days post-tumor cell injection. Mice were sacrificed after 4 weeks post-tumor cell injection. In all experiments, n=3-8 per group. ns, not significant. *p≤0.05, **p≤0.01, ***p≤0.001 and ****p≤0.0001.

### Example 9. Diagnosis and Treatment of Cachexia

Increased Zip14-mediated zinc accumulation can promote cancer-induced muscle wasting. Zip14-mediated zinc uptake can block muscle-cell differentiation and induce myosin heavy chain loss. Since both processes contribute to muscle atrophy in metastatic cancers, monitoring zinc consumption in metastatic cancer patients using Zip 14 as a biomarker can provide a method to diagnose the development of cachexia.

Zip14 can be used as a therapeutic target for treating cancer-induced cachexia. A method for treating cachexia includes administering a zinc chelating agent to a patient to reduce the zinc level in patient's muscle, wherein the zinc chelating agent includes: 1,10-phenanthroline, N,N,N′,N′-tetrakis(2-pyridylmethyl) ethane-1,2-diamine (TPEN), clioquinol (5-chloro-7-iodo-quinolin-8-ol), DEDTC (sodium diethyldithiocarbamate trihydrate), DTPA (diethylene triamine pentaacetic acid), EDDA (ethylenediamine-N,N′-diacetic acid), and EDTA (ethylenediaminetetraacetic acid) described in Laskaris; the pyrrolyi-hydroxamates described in WO2013/182254 A1 (Valenti et al., Pyrrolyi-hydroxamates for use in the prevention and/or treatment and/or treatment of bacterial infection; WO2013/182254 A1 is incorporated herein by reference); the zinc chelating agents described in U.S. Pat. No. 9,320,736 B2 (Kutikov et al., Zinc chelating agents for depleting XIAP and sensitizing tumor cells to apoptosis; U.S. Pat. No. 9,320,736 B2 is incorporated herein by reference); and the nanoparticles described in US 2014/0303081 A1 (Dhar et al., Apoptosis-targeting nanoparticles; US 2014/0303081 A1 is incorporated herein by reference). In addition to administering a zinc chelating agent, the method of treating cachexia may further include restricting zinc uptakes in patient's diet, such as providing zinc-free water, zinc-free food or combinations thereof.

In addition to administering a zinc chelating agent, the method of treating cachexia may further include administering a muscle-specific targeting agent. The muscle-specific targeting agent includes: the nanoparticles and conjugates described in WO2015/116565 A2 (Daftarian et al., Muscle cell-targeting nanoparticles for vaccination and nucleic acid delivery, and methods of production and use thereof; WO2015/116568 A2 is incorporated herein by reference); the rapamycin-loaded nanoparticles described in U.S. Pat. No. 9,412,018 B2 (Wickline et al., Methods for improving muscle strength; U.S. Pat. No. 9,412,018 B2 is incorporated herein by reference); the nanoparticle that incorporated peptides described in U.S. Pat. No. 9,486,409 B2 (Edelson et al., Peptide nanoparticles and uses thereof; U.S. Pat. No. 9,486,409 B2 is incorporated herein by reference); and the nanoparticles described in EP2488165 B1 (Ferlini et al., Nanoparticle of the core-shell type suitable for delivering therapeutic oligonucleotides to target tissues and the use thereof for the preparation of a medicament for treating Duchenne muscular dystrophy; EP2488165 B1 is incorporated herein by reference).

A method for treating cachexia includes administering an inhibitor of the Zip 14 protein to a patient to reduce the zinc level in patient's muscle, wherein the inhibitor of the Zip 14 protein includes an antagonist of Zip14 protein.

A method for treating cachexia includes administering a nucleic acid to a patient to reduce or eliminate the expression of Zip14 in patient's muscle, wherein the nucleic acid includes short hairpin RNA (shRNA), short interfering RNA (siRNA), or a nucleic acid for gene editing.

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention as defined by the appended claims.

### REFERENCES

1. Hanahan, D. & Weinberg, R. A. Hallmarks of cancer: the next generation. Cell 144, 646-674 (2011).
2. Lambert, A. W., Pattabiraman, D. R. & Weinberg, R. A. Emerging Biological Principles of Metastasis. Ce//168, 670-691 (2017).
3. Massague, J. & Obenauf, A. C. Metastatic colonization by circulating tumour cells. Nature 529, 298-306 (2016).
4. Wan, L., Pantel, K. & Kang, Y. Tumor metastasis: moving new biological insights into the clinic. Nature medicine 19, 1450-1464 (2013).
5. Loberg, R. D., Bradley, D. A., Tomlins, S. A., Chinnaiyan, A. M. & Pienta, K. J. The lethal phenotype of cancer: the molecular basis of death due to malignancy. CA Cancer J Clin 57, 225-241 (2007).
6. Waning, D. L., et al. Excess TGF-beta mediates muscle weakness associated with bone metastases in mice. Nature medicine (2015).
7. Becker, A., et al. Extracellular Vesicles in Cancer: Cell-to-Cell Mediators of Metastasis. Cancer cell 30, 836-848 (2016).
8. Argiles, J. M., Busquets, S., Stemmier, B. & Lopez-Soriano, F. J. Cancer cachexia: understanding the molecular basis. Nature reviews. Cancer 14, 754-762 (2014).
9. Fearon, K. C., Glass, D. J. & Guttridge, D. C. Cancer cachexia: mediators, signaling, and metabolic pathways. Cell metabolism 16, 153-166 (2012).
10. Fearon, K., Arends, J. & Baracos, V. Understanding the mechanisms and treatment options in cancer cachexia. Nature reviews. Clinical oncology 10, 90-99 (2013).
11. Cohen, S., Nathan, J. A. & Goldberg, A. L. Muscle wasting in disease: molecular mechanisms and promising therapies. Nat Rev Drug Discov 14, 58-74 (2015).
12. Sandri, M. Protein breakdown in cancer cachexia. Seminars in cell & developmental biology 54, 11-19 (2016).
13. Bozzetti, F. & Group, S. W. Screening the nutritional status in oncology: a preliminary report on 1,000 outpatients. Supportive care in cancer: official journal of the Multinational Association of Supportive Care in Cancer 17, 279-284 (2009).
14. Penna, F., Busquets, S. & Argiles, J. M. Experimental cancer cachexia: Evolving strategies for getting closer to the human scenario. Seminars in cell & developmental biology 54, 20-27 (2016).
15. Hara, T., et al. Physiological roles of zinc transporters: molecular and genetic importance in zinc homeostasis. J Physiol Sci 67, 283-301 (2017).
16. Lichten, L. A. & Cousins, R. J. Mammalian zinc transporters: nutritional and physiologic regulation. Annu Rev Nutr 29, 153-176 (2009).
17. Finney, L. A. & O'Halloran, T. V. Transition metal speciation in the cell: insights from the chemistry of metal ion receptors. Science 300, 931-936 (2003).
18. Larsson, S., Karlberg, I., Selin, E., Daneryd, P. & Peterson, H.l. Trace element changes in serum and skeletal muscle compared to tumour tissue in sarcoma-bearing rats. In Vivo 1, 131-140 (1987).
19. Russell, S. T., Siren, P. M., Siren, M. J. & Tisdale, M. J. The role of zinc in the anti-tumour and anti-cachectic activity of D-myo-inositol 1,2,6-triphosphate. British journal of cancer 102, 833-836 (2010).
20. Siren, P. M. & Siren, M. J. Systemic zinc redistribution and dyshomeostasis in cancer cachexia. Journal of cachexia, sarcopenia and muscle 1, 23-33 (2010).
21. Talmadge, J. E. & Fidler, I. J. Enhanced metastatic potential of tumor cells harvested from spontaneous metastases of heterogeneous murine tumors. Journal of the National Cancer Institute 69, 975-980 (1982).
22. Kang, Y., et al. A multigenic program mediating breast cancer metastasis to bone. Cancer cell 3, 537-549 (2003).
23. Francia, G., Cruz-Munoz, W., Man, S., Xu, P. & Kerbel, R. S. Mouse models of advanced spontaneous metastasis for experimental therapeutics. Nature reviews. Cancer 11, 135-141 (2011).
24. Bodine, S. C., et al. Identification of ubiquitin ligases required for skeletal muscle atrophy. Science 294, 1704-1708 (2001).
25. Santra, M. K., Wajapeyee, N. & Green, M. R. F-box protein FBX031 mediates cyclin D1 degradation to induce G1 arrest after DNA damage. Nature 459, 722-725 (2009).
26. Sartori, R., et al. BMP signaling controls muscle mass. Nat Genet 45, 1309-1318 (2013).
27. Milan, G., et al. Regulation of autophagy and the ubiquitin-proteasome system by the FoxO transcriptional network during muscle atrophy. Nat Commun 6, 6670 (2015).
28. Azoulay, E., et al. The prognosis of acute respiratory failure in critically ill cancer patients. Medicine (Baltimore) 83, 360-370 (2004).
29. Fearon, K. C. Cancer cachexia: developing multimodal therapy for a multidimensional problem. Eur J Cancer 44, 1124-1132 (2008).
30. Iguchi, H., Onuma, E., Sato, K., Sato, K. & Ogata, E. Involvement of parathyroid hormone-related protein in experimental cachexia induced by a human lung cancer-derived cell line established from a bone metastasis specimen. International journal of cancer. Journal international du cancer 94, 24-27 (2001).
31. Shum, A. M., et al. Cardiac and skeletal muscles show molecularly distinct responses to cancer cachexia. Physiol Genomics 47, 588-599 (2015).
32. Bonetto, A., et al. STAT3 activation in skeletal muscle links muscle wasting and the acute phase response in cancer cachexia. PloS one 6, e22538 (2011).
33. Kwon, M. C. & Berns, A. Mouse models for lung cancer. Mol Oncol 7, 165-177 (2013).
34. Ji, H., et al. LKB1 modulates lung cancer differentiation and metastasis. Nature 448, 807-810 (2007).
35. Nguyen, D. X., et al. WNT/TCF signaling through LEF1 and HOXB9 mediates lung adenocarcinoma metastasis. Cell 138, 51-62 (2009).
36. Meuwissen, R., et al. Induction of small cell lung cancer by somatic inactivation of both Trp53 and Rb1 in a conditional mouse model. Cancer cell 4, 181-189 (2003).
37. Schaffer, B. E., et al. Loss of p130 accelerates tumor development in a mouse model for human small-cell lung carcinoma. Cancer research 70, 3877-3883 (2010).
38. Cai, D., et al. IKKbeta/NF-kappaB activation causes severe muscle wasting in mice. Cell 119, 285-298 (2004).
39. Guttridge, D. C., Mayo, M. W., Madrid, L. V., Wang, C. Y. & Baldwin, A. S., Jr. NF-kappaB-induced loss of MyoD messenger RNA: possible role in muscle decay and cachexia. Science 289, 2363-2366 (2000).
40. Zimmers, T. A., et al. Induction of cachexia in mice by systemically administered myostatin. Science 296, 1486-1488 (2002).
41. Zhou, X., et al. Reversal of cancer cachexia and muscle wasting by ActRIIB antagonism leads to prolonged survival. Cell 142, 531-543 (2010).
42. Hojyo, S., et al. The zinc transporter SLC39A14/ZIP14 controls G-protein coupled receptor-mediated signaling required for systemic growth. PloS one 6, e18059 (2011).
43. Liuzzi, J. P., et al. Interleukin-6 regulates the zinc transporter Zip14 in liver and contributes to the hypozincemia of the acute-phase response. Proceedings of the National Academy of Sciences of the United States of America 102, 6843-6848 (2005).
44. Gee, K. R., Zhou, Z. L., Qian, W. J. & Kennedy, R. Detection and imaging of zinc secretion from pancreatic beta-cells using a new fluorescent zinc indicator. Journal of the American Chemical Society 124, 776-778 (2002).
45. He, W. A., et al. NF-kappaB-mediated Pax7 dysregulation in the muscle microenvironment promotes cancer cachexia. The Journal of clinical investigation 123, 4821-4835 (2013).
46. Sabourin, L. A. & Rudnicki, M. A. The molecular regulation of myogenesis. Clin Genet 57, 16-25 (2000).
47. Penna, F., et al. Muscle wasting and impaired myogenesis in tumor bearing mice are prevented by ERK inhibition. PloS one 5, e13604 (2010).
48. Borisov, A. B., Dedkov, E.l. & Carlson, B. M. Abortive myogenesis in denervated skeletal muscle: differentiative properties of satellite cells, their migration, and block of terminal differentiation. Anat Embryol (Berl) 209, 269-279 (2005).
49. Acharyya, S., et al. Dystrophin glycoprotein complex dysfunction: a regulatory link between muscular dystrophy and cancer cachexia. Cancer cell 8, 421-432 (2005).
50. Zhang, L., Wang, X. H., Wang, H., Du, J. & Mitch, W. E. Satellite cell dysfunction and impaired IGF-1 signaling cause CKD-induced muscle atrophy. Journal of the American Society of Nephrology: JASN 21, 419-427 (2010).
51. Langen, R. C., et al. Muscle wasting and impaired muscle regeneration in a murine model of chronic pulmonary inflammation. Am J Respir Cell Mol Biol 35, 689-696 (2006).
52. Liu, N., et al. Requirement of MEF2A, C, and D for skeletal muscle regeneration. Proceedings of the National Academy of Sciences of the United States of America 111, 4109-4114 (2014).
53. Skapek, S. X., Rhee, J., Spicer, D. B. & Lassar, A. B. Inhibition of myogenic differentiation in proliferating myoblasts by cyclin D1-dependent kinase. Science 267, 1022-1024 (1995).
54. Wei, Q. & Paterson, B. M. Regulation of MyoD function in the dividing myoblast. FEBS Lett 490, 171-178 (2001).
55. Eley, H. L., Skipworth, R. J., Deans, D. A., Fearon, K. C. & Tisdale, M. J. Increased expression of phosphorylated forms of RNA-dependent protein kinase and eukaryotic initiation factor 2alpha may signal skeletal muscle atrophy in weight-losing cancer patients. British journal of cancer 98, 443-449 (2008).
56. Acharyya, S., et al. Cancer cachexia is regulated by selective targeting of skeletal muscle gene products. The Journal of clinical investigation 114, 370-378 (2004).
57. Glass, D. J. Signaling pathways perturbing muscle mass. Current opinion in clinical nutrition and metabolic care 13, 225-229 (2010).
58. Roberts, B. M., et al. Diaphragm and ventilatory dysfunction during cancer cachexia. FASEB journal: official publication of the Federation of American Societies for Experimental Biology 27, 2600-2610 (2013).
59. Cohen, S., et al. During muscle atrophy, thick, but not thin, filament components are degraded by MuRF1-dependent ubiquitylation. The Journal of cell biology 185, 1083-1095 (2009).
60. Clarke, B. A., et al. The E3 Ligase MuRF1 degrades myosin heavy chain protein in dexamethasone-treated skeletal muscle. Cell metabolism 6, 376-385 (2007).
61. Gupta, S. K., Shukla, V. K., Vaidya, M. P., Roy, S. K. & Gupta, S. Serum and tissue trace elements in colorectal cancer. J Surg Oncol 52, 172-175 (1993).
62. Gupta, S. K., Shukla, V. K., Vaidya, M. P., Roy, S. K. & Gupta, S. Serum trace elements and Cu/Zn ratio in breast cancer patients. J Surg Oncol 46, 178-181 (1991).
63. Fabris, C., et al. Copper, zinc and copper/zinc ratio in chronic pancreatitis and pancreatic cancer. Clin Biochem 18, 373-375 (1985).
64. Cousins, R. J. & Leinart, A. S. Tissue-specific regulation of zinc metabolism and metallothionein genes by interleukin 1. FASEB journal: official publication of the Federation of American Societies for Experimental Biology 2, 2884-2890 (1988).
65. Summermatter, S., et al. Blockade of Metallothioneins 1 and 2 Increases Skeletal Muscle Mass and Strength. Molecular and cellular biology 37(2017).
66. Crawford, A. J. & Bhattacharya, S. K. Excessive intracellular zinc accumulation in cardiac and skeletal muscles of dystrophic hamsters. Exp Neurol 95, 265-276 (1987).
67. Lecker, S. H., et al. Multiple types of skeletal muscle atrophy involve a common program of changes in gene expression. FASEB journal: official publication of the Federation of American Societies for Experimental Biology 18, 39-51 (2004).
68. Balkwill, F. TNF-alpha in promotion and progression of cancer. Cancer metastasis reviews 25, 409-416 (2006).
69. Kang, Y., et al. Breast cancer bone metastasis mediated by the Smad tumor suppressor pathway. Proceedings of the National Academy of Sciences of the United States of America 102, 13909-13914 (2005).
70. Jenkitkasemwong, S., et al. SLC39A14 Is Required for the Development of Hepatocellular Iron Overload in Murine Models of Hereditary Hemochromatosis. Cell metabolism 22, 138-150 (2015).
71. Schiaffino, S., Dyar, K. A., Ciciliot, S., Blaauw, B. & Sandri, M. Mechanisms regulating skeletal muscle growth and atrophy. FEBS J 280, 4294-4314 (2013).
72. Schmitt, T. L., et al. Activity of the Akt-dependent anabolic and catabolic pathways in muscle and liver samples in cancer-related cachexia. J Mol Med (Bed) 85, 647-654 (2007).
73. Yamasaki, S., et al. Zinc is a novel intracellular second messenger. The Journal of cell biology 177, 637-645 (2007).
74. Andreini, C., Bertini, I. & Rosato, A. Metalloproteomes: a bioinformatic approach. Acc Chem Res 42, 1471-1479 (2009).
75. Dumont, N. A., Wang, Y. X. & Rudnicki, M. A. Intrinsic and extrinsic mechanisms regulating satellite cell function. Development 142, 1572-1581 (2015).
76. Talbert, E. E. & Guttridge, D. C. Impaired regeneration: A role for the muscle microenvironment in cancer cachexia. Seminars in cell & developmental biology 54, 82-91 (2016).
77. Wallace, G. Q. & McNally, E. M. Mechanisms of muscle degeneration, regeneration, and repair in the muscular dystrophies. Anna Rev Physiol 71, 37-57 (2009).
78. Hood, M.l. & Skaar, E. P. Nutritional immunity: transition metals at the pathogen-host interface. Nat Rev MicrobionO, 525-537 (2012).
79. Vallee, B. L. & Falchuk, K. H. The biochemical basis of zinc physiology. Physiological reviews 73, 79-118 (1993).
80. Talmadge, J. E. & Fidler, I. J. Enhanced metastatic potential of tumor cells harvested from spontaneous metastases of heterogeneous murine tumors. Journal of the National Cancer Institute 69, 975-980 (1982).
81. Acharyya, S., et al. Dystrophin glycoprotein complex dysfunction: a regulatory link between muscular dystrophy and cancer cachexia. Cancer cell 8, 421-432 (2005).
82. Blanco, M. A., et al. Global secretome analysis identifies novel mediators of bone metastasis. Cell research 22, 1339-1355 (2012).
83. Nguyen, D. X., et al. WNT/TCF signaling through LEF1 and HOXB9 mediates lung adenocarcinoma metastasis. Cell 138, 51-62 (2009).
84. Jahchan, N. S., et al. A drug repositioning approach identifies tricyclic antidepressants as inhibitors of small cell lung cancer and other neuroendocrine tumors. Cancer discovery 3, 1364-1377 (2013).
85. Dalai, B. I., Keown, P. A. & Greenberg, A. H. Immunocytochemical localization of secreted transforming growth factor-beta 1 to the advancing edges of primary tumors and to lymph node metastases of human mammary carcinoma. The American journal of pathology 143, 381-389 (1993).
86. Hojyo, S., et al. The zinc transporter SLC39A14/ZIP14 controls G-protein coupled receptor-mediated signaling required for systemic growth. PloS one 6, e18059 (2011).
87. Jenkitkasemwong, S., et al. SLC39A14 Is Required for the Development of Hepatocellular Iron Overload in Murine Models of Hereditary Hemochromatosis. Cell metabolism 22, 138-150 (2015).
88. Burkholder, T., Foltz, C., Karlsson, E., Linton, C. G. & Smith, J. M. Health Evaluation of Experimental Laboratory Mice. Curr Protoc Mouse Biol 2, 145-165 (2012).
89. DuPage, M., Dooley, A. L. & Jacks, T. Conditional mouse lung cancer models using adenoviral or lentiviral delivery of Cre recombinase. Nat Protoc 4, 1064-1072 (2009).
90. Acharyya, S., et at. Cancer cachexia is regulated by selective targeting of skeletal muscle gene products. J Clin Invest 114, 370-378 (2004).
91. Francia, G., Cruz-Munoz, W., Man, S., Xu, P. & Kerbel, R. S. Mouse models of advanced spontaneous metastasis for experimental therapeutics. Nature reviews. Cancer 11, 135-141 (2011).
92. Acharyya, S., et al. A CXCL1 paracrine network links cancer chemoresistance and metastasis. Cell 150, 165-178 (2012).
93. Livak, K. J. & Schmittgen, T. D. Analysis of relative gene expression data using real-time quantitative PCR and the 2(−Delta C(T)) Method. Methods 25, 402-408 (2001).
94. Liberzon, A., et al. The Molecular Signatures Database (MSigDB) hallmark gene set collection. Cell SysM, 417-425 (2015).
95. Subramanian, A., et al. Gene set enrichment analysis: a knowledge-based approach for interpreting genome-wide expression profiles. Proceedings of the National Academy of Sciences of the United States of America 102, 15545-15550 (2005).
96. Ohly, P., Dohle, C., Abel, J., Seissler, J. & Gleichmann, H. Zinc sulphate induces metallothionein in pancreatic islets of mice and protects against diabetes induced by multiple low doses of streptozotocin. Diabetologia 43, 1020-1030 (2000).
97. Buclez, P. O., et al. Rapid, scalable, and low-cost purification of recombinant adeno-associated virus produced by baculovirus expression vector system. Molecular therapy. Methods & clinical development 3, 16035 (2016).
98. Wahlen, R., Evans, L., Turner, J. & Hearn, R. The use of collision/reaction cell ICP-MS for the determination of elements in blood and serum samples. Spectroscopy 20, 84-89 (2005).
99. Vogler, T. O., Gadek, K. E., Cadwallader, A. B., Elston, T. L. & Olwin, B. B. Isolation, Culture, Functional Assays, and Immunofluorescence of Myofiber-Associated Satellite Cells. Methods in molecular biology 1460, 141-162 (2016).
100. Motohashi, N., Asakura, Y. & Asakura, A. Isolation, culture, and transplantation of muscle satellite cells. J Vis Exp (2014).
101. He, W. A., et al. NF-kappaB-mediated Pax7 dysregulation in the muscle microenvironment promotes cancer cachexia. The Journal of clinical investigation 123, 4821-4835 (2013).
102. Volodin, A., Kosti, I., Goldberg, A. L. & Cohen, S. Myofibril breakdown during atrophy is a delayed response requiring the transcription factor PAX4 and desmin depolymerization. Proceedings of the National Academy of Sciences of the United States of America 114, E1375-E1384 (2017).
103. Acharyya, S., et al. Cancer cachexia is regulated by selective targeting of skeletal muscle gene products. The Journal of clinical investigation 114, 370-378 (2004).
104. Cosper, P. F. & Leinwand, L. A. Myosin heavy chain is not selectively decreased in murine cancer cachexia. International journal of cancer. Journal international du cancer 130, 2722-2727 (2012).
105. Roberts, B. M., et al. Diaphragm and ventilatory dysfunction during cancer cachexia. FASEB journal official publication of the Federation of American Societies for Experimental Biology 27, 2600-2610 (2013).

What is claimed is:

1. A method for treating cachexia in a patient which comprises reducing bioavailable zinc in the patient to reduce, inhibit or prevent zinc accumulation in the patient's muscle.

2. The method of claim 1 wherein the bioavailable zinc is reduced by administering a zinc chelating agent to the patient.

3. The method of claim 2 wherein the cachexia is induced by cancer and the zinc chelating agent is administered along with, just prior to or immediately after the administration of a cancer treating drug.

4. The method of claim 2 wherein the zinc chelating agent is present in the pharmaceutical composition in an effective amount to reduce Zip14-mediated zinc accumulation in the patient's muscle.

5. The method of claim 4, wherein the cachexia is induced by cancer and the pharmaceutical composition is administered along with, just prior to or immediately after the administration of a cancer treating drug.

6. The method of claim 4, wherein the zinc chelating agent is present in the pharmaceutical composition in an effective amount to reduce loss of myosin heavy chain in the patient's muscle.

7. The method of claim 4, wherein the zinc chelating agent is present in the pharmaceutical composition in an effective amount to promote muscle-cell differentiation in the patient's muscle.

8. The method of claim 4, wherein the zinc chelating agent is present in the pharmaceutical composition that further comprises a muscle-specific targeting agent.

9. The method of claim 1, wherein the method further comprises restricting zinc uptake from the patient's diet.

10. The method of claim 1, wherein the bioavailable zinc is reduced by administering an inhibitor of a Zip14 protein to the patient.

11. The method of claim 10, wherein the inhibitor of the Zip14 protein is an antagonist of the Zip14 protein.

12. The method of claim 1, wherein the bioavailable zinc is reduced by administering a nucleic acid which is reduces or eliminates the expression of Zip14 in the patient's muscle.

13. The method of claim 12, wherein the nucleic acid is a short hairpin RNA, a short interfering RNA, or a nucleic acid for gene editing.

14. A method for diagnosing the development or progression of cachexia in a patient which comprises monitoring zinc accumulation in the patient's muscle.

15. The method of claim 14, wherein the zinc accumulation is Zip14-mediated zinc accumulation, and wherein the cachexia is induced by cancer.

16. The method of claim 15, which further comprises monitoring the expression level of Zip14 in the patient's muscle.

17. The method of claim 14, which further comprises monitoring loss of myosin heavy chain in the patient's muscle.

18. The method of claim 14, which further comprises monitoring a reduction of muscle-cell differentiation in the patient's muscle.

19. A method for monitoring a development or a progression of cachexia in a patient using Zip14 as a biomarker, which comprises detecting an increased-level of Zip14 protein in the patient or by detecting an increased-level of Zip14-mediated zinc accumulation in the patient's muscle.

20. The method of claim 19, wherein the development or progression of cachexia is reduced or inhibited by administering an inhibitor of a Zip14 protein to the patient.


SEQUENCE LISTING (9 sequences)

SEQ ID NO: 1 - DNA, 21 bp, Artificial Sequence
  AAV9 Forward Primer
ttacggtaaactgcccacttg

SEQ ID NO: 2 - DNA, 24 bp, Artificial Sequence
  AAV9 Reverse Primer
cataaggtcatgtactgggcataa

SEQ ID NO: 3 - DNA, 61 bp, Artificial Sequence
  Zip14 shRNA sequence
ccgggcaggctctcttcttcaacttctcgcgaagttgaagaagaagagagcctgctttttg

SEQ ID NO: 4 - protein, 36 aa, Mus musculus
KQKNEHHHGHNHFTSETLPSKKDQEEGVTEKLQNGD

SEQ ID NO: 5 - protein, 36 aa, Homo sapiens
KQKNEHHHGHSHYASESLPSKKDQEEGVMEKLQNGD

SEQ ID NO: 6 - protein, 36 aa, Mus musculus
LDHMIPQHCNSELDGKAPGTDEKVIVNSMSVQDLQA

SEQ ID NO: 7 - protein, 36 aa, Homo sapiens
LDHMIPQHCSSELDGKAPMVDEKVIVGSLSVQDLQA

SEQ ID NO: 8 - protein, 35 aa, Mus musculus
SQSACYWLKGVRYSDIGTLAWMITLSDGLHNFIDG

SEQ ID NO: 9 - protein, 35 aa, Homo sapiens; identical to SEQ ID NO: 8